(19) United States
(12) Patent Application Publication
(10) Pub. No.: US 2015/0023876 A1
(43) Pub. Date: Jan. 22, 2015

(54) COMPOSITIONS, METHODS AND KITS FOR DIAGNOSING AND TREATING CD206 EXPRESSING CELL-RELATED DISORDERS

(71) Applicants: NAVIDEA BIOPHARMACEUTICALS, INC., Dublin, OH (US); OHIO STATE INNOVATION FOUNDATION, Columbus, OH (US)

(72) Inventors: Frederick O. Cope, Dublin, OH (US); Michael S. Blue, Dublin, OH (US); Wendy L. Metz, Dublin, OH (US); Larry S. Schlesinger, Columbus, OH (US)

(21) Appl. No.: 14/338,332

(22) Filed: Jul. 22, 2014

Related U.S. Application Data
(60) Provisional application No. 61/857,232, filed on Jul. 22, 2013.
(60) Provisional application No. 61/879,649, filed on Sep. 18, 2013.

(51) Int. Cl. A61K 51/04; G01N 33/569; A61K 49/00

(52) U.S. Cl. CPC A61K 51/0491; A61K 49/0054; G01N 33/56966; G01N 2333/70596
USPC 424/1.73; 424/9.1; 424/9.6; 435/7.21

(57) ABSTRACT

A method of diagnosing a CD206 expressing cell-related disorder by administering a pharmaceutical composition to a subject, the composition including a carrier molecule having a detectable moiety attached thereto. The carrier molecule has a dextran backbone, and at least one receptor substrate conjugated, directly or indirectly, to the dextran backbone, wherein the receptor substrate is chosen so as to specifically bind to CD206. A method of treating a CD206 expressing cell-related disorder is also provided, as well as an ex vivo method and kit for quantitating the number of cells expressing CD206 in a bodily fluid.

### CROSS REFERENCE TO RELATED APPLICATIONS

This application claims priority to U.S. Provisional Patent Application Nos. 61/857,232 filed on Jul. 22, 2013, entitled “COMPOSITIONS AND METHODS FOR DIAGNOSING AND TREATING MACROPHAGE-RELATED DISORDERS,” and 61/879,649, filed on Sep. 18, 2013, entitled “COMPOSITIONS AND METHODS FOR DIAGNOSING AND TREATING MACROPHAGE-RELATED DISORDERS.” The entire disclosures of the foregoing provisional patent applications are incorporated by reference herein.

### BACKGROUND

Various receptor-binding compounds have been developed for use in the diagnosis or treatment of various medical conditions. Such receptor-binding compounds typically are designed to bind to one or more receptor sites on one or more specific proteins. Receptor-binding compounds can be used to deliver therapeutic or diagnostic agents to specific target cells, or even to block certain receptors for therapeutic reasons.

By way of example, U.S. Pat. No. 6,409,990 (“the '990 patent”), titled “Macromolecular Carrier for Drug and Diagnostic Agent Delivery,” which issued on Jun. 25, 2002 and is incorporated herein by way of reference, discloses receptor-binding macromolecules which have been shown to be useful as carrier molecules for the delivery of radioisotopes for use in sentinel node imaging for staging breast cancer and melanoma. The carrier molecules described in the '990 patent exhibit significant and sustained uptake by sentinel lymph nodes, thus allowing the delivery of the radioisotopes attached to the carrier molecule.

By way of a more specific example, one currently marketed diagnostic agent produced in accordance with the '990 patent is technetium Tc 99m tilmanocept, which is marketed by Navidea Biopharmaceuticals Inc. of Dublin, Ohio, under the name LYMPHOSEEK® Injection kit. The LYMPHOSEEK kit is distributed in the form of vials containing tilmanocept powder. The tilmanocept powder is radiolabeled with technetium Tc 99m prior to use in order to prepare the technetium Tc 99m tilmanocept diagnostic agent. This diagnostic agent is formed when a technetium Tc 99m pertechnetate solution is added to the vial containing the tilmanocept powder, and a reducing agent, such that the technetium Tc 99m is chelated to the diethylenetriaminepentaacetic acid (“DTPA”) moieties of the tilmanocept molecule. The resulting radioactive diagnostic agent is approved for use in lymphatic mapping using single-photon emission computerized tomography (SPECT; with or without computerized tomography, CT), and/or gamma-emission-based scintigraphy, and/or using a hand-held gamma counter in order to assist in the localization of lymph nodes draining a primary tumor site (i.e., sentinel lymph nodes) in patients having breast cancer, melanoma, or squamous cell carcinoma (SCC).

Tilmanocept, the non-radiolabeled precursor of the LYMPHOSEEK® diagnostic agent, has a dextran backbone to which a plurality of amino-terminated leashes (—O(CH₂)₃S(CH₂)₂NH₂) are attached to the core glucose elements. In addition, mannose moieties are conjugated to amino groups of a number of the leashes, and the chelator diethylenetriamine pentaacetic acid (DTPA) is conjugated to the amino group of other leashes not containing the mannose. Tilmanocept generally consists of dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes, and generally has the following structure:

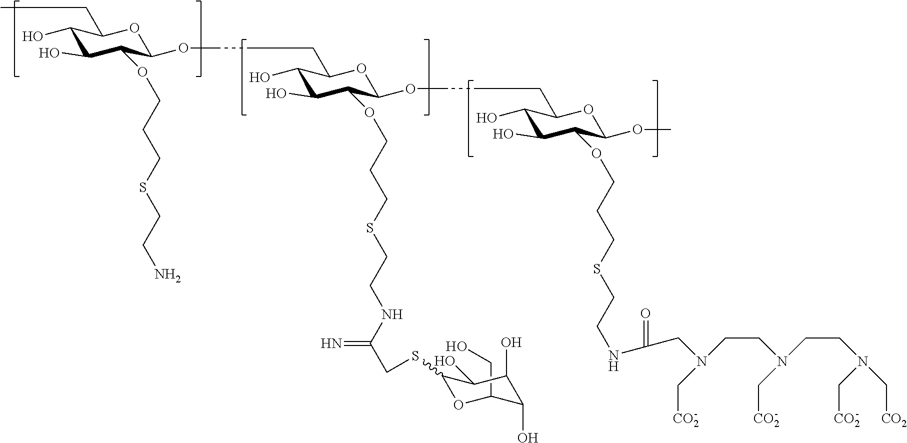

It should be noted that in some instances certain ones of the glucose moieties may have no attached aminothiol leash.

The DTPA chelator portion of tilmanocept is used for attachment of the radioactive isotope Tc 99m to the carrier molecule. After radiolabeling (e.g., as described in the '990 patent), technetium tilmanocept is formed: technetium Tc 99m, dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes. Technetium Tc 99m tilmanocept has the following structure:

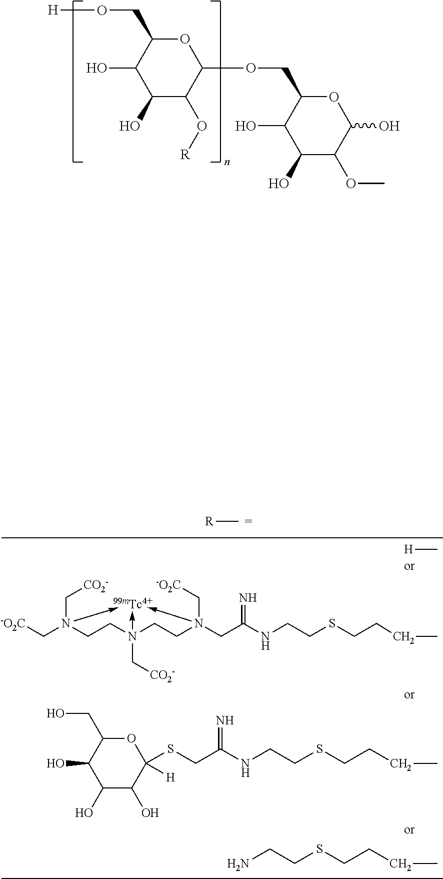

The molecular formula of technetium Tc 99m tilmanocept is [C₆H₁₀O₅]ₙ.(C₁₉H₂₈N₄O₉S^{99m}TC)ₐ.(C₁₃H₂₄N₂O₅S₂)_{b}.(C₅H₁₁NS)_{c}, wherein n is between about 35 and about 58, and n≧(a+b+c). In the commercially marketed version, it contains 3-8 conjugated DTPA (diethylenetriamine pentaacetic acid) moieties (a); 12-20 conjugated mannose moieties (b), and 0-17 unconjugated amine side chains (c).

When used to stage breast cancer, melanoma or SCC, technetium Tc 99m labeled tilmanocept (i.e., Lymphoseek) demonstrates rapid clearance from an injection site, rapid and sustained uptake by the sentinel lymph node(s), and low uptake by distal or second-echelon lymph nodes. While the mannose moiety on tilmanocept was known to be responsible for receptor binding, the nature and scope of such binding was not known.

While a variety of devices and techniques may exist for diagnosing and/or treating macrophage related disorders, it is believed that no one prior to the inventor(s) has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings.

FIG. 1A depicts fluorescence-activated cell sorting (“FACS”) analysis of PBMC cells incubated with Cy3-labeled tilmanocept.

FIG. 1B depicts FACS analysis of PBMC cells incubated with Cy3-tilmanocept, with and without pre-treatment with cold tilmanocept.

FIG. 1C depicts microscopy images of MDM cells treated tilmanocept, wherein the upper and lower left images in FIG. 1C depict confocal microscopy representative images showing binding (upper left) and inhibition of binding (lower left) of tilmanocept-Cy3 to macrophages in the absence or presence of pre-treatment with cold tilmanocept and the upper and lower right images in FIG. 1C are DIC images which show the individual cell structure of the adjacent fluorescent images (to the left of each DIC image).

FIG. 2 depicts confocal images of MDM monolayers showing expression of CD206 (FIG. 2A), tilmanocept binding by the macrophage (FIG. 2B), and co-localization between CD206 and tilmanocept in both confocal and phase contrast images (FIGS. 2C and 2D).

FIG. 3 depicts a photomicrograph of KS tumor cells showing markers for nuclei, KS tumor cells and CD206.

FIG. 4A depicts KS tumor cells and macrophages expressing CD206 and binding to tilmanocept-Cy3 on the surface, and FIG. 4B depicts the internalization of tilmanocept-Cy3 into cytoplasmic vesicles.

FIG. 5 depicts confocal microscopy representative images showing co-localization of macrophage mannose receptor CD206 on both LANA expressing KS tumor cells and tissue macrophages.

FIG. 6 depicts confocal images of KS biopsy tissue culture with Cy3 tilmanocept.

FIG. 7 depicts a flow cytometric evaluation of Cy3 and CD206 in 3 day CD206+ macrophage cultures incubated with Cy3-tilmanocept.

FIG. 8 depicts confocal microscopy images of TB-infected macrophages exposed to Cy3-tilmanocept.

FIG. 9 depicts immunofluorescent staining of left ventricle and aorta from rhesus macaques, illustrating the co-localization of CD163/Alex-Fluor 488, CD206/Alexa-Fluor 568, and Cy3 tilmanocept.

FIG. 10 depicts the results of a comparison of the binding of Cy3-tilmanocept to RA, OA and normal synovial tissue and fluid.

FIG. 11 depicts the results of a comparison of Cy3-tilmanocept binding in vivo in the knees and elbows of cartilage antibody-induced arthritic mice and a control.

FIG. 12 depicts in vivo fluorescence of the elbows and feet of a mouse with immune-mediated arthritis (top) and control mouse (bottom).

FIG. 13 depicts the results of a comparison of Cy3-tilmanocept binding ex vivo in the knees and elbows of arthritic mice and a control.

FIG. 14 depicts ex vivo fluorescence of the knees of control mice and mice with immune-mediated arthritis.

### DETAILED DESCRIPTION

The following description of certain examples should not be used to limit the scope of the present invention. Other features, aspects, and advantages of the versions disclosed herein will become apparent to those skilled in the art from the following description. As will be realized, the versions described herein are capable of other different and obvious aspects, all without departing from the invention. Accordingly, the drawings and descriptions should be regarded as illustrative in nature and not restrictive.

The present invention is directed to compositions, methods and kits for the diagnosis and/or treatment of CD206 expressing cell-related disorders using synthetic macromolecules (e.g., about 2-30 kDa). The CD-206 expressing cell-related disorders include any disease, disorder or condition in which macrophages, dendritic cells or other CD206 expressing cells are involved or recruited, such as those in which the number of macrophages or other CD206 expressing cells is increased and/or such cells are abnormally localized (e.g., in tumors, in affected joints, to vascular endothelium, etc.). Such disorders include, but are not limited to, immune diseases, immune-mediated immune diseases, autoimmune diseases, inflammatory diseases, auto-inflammatory diseases, and infectious diseases.

As further discussed below, the compositions described herein include synthetic, macromolecular carrier molecules, as well as synthetic, macromolecular carrier molecules having one or more detectable moieties and/or therapeutic agents attached thereto. Embodiments described herein also provide diagnostic and/or treatment kits containing such carrier molecules, optionally in a pharmaceutically acceptable carrier (e.g., one which includes a pharmaceutically acceptable vehicle) suitable for administering the carrier molecule to a mammalian subject, or in a solution which facilitates ex vivo diagnostic testing. In some embodiments the kit comprises a carrier molecule in a form suitable for attaching one or more detectable moieties and/or one or more therapeutic agents to the carrier molecule, while in other embodiments the kit comprises the carrier molecule already having one or more detectable moieties and/or one or more therapeutic agents attached thereto. In one particular embodiment, a kit comprises the carrier molecule (e.g., in the form of a lyophilized powder) in a container along with one or more adjuvants for facilitating the attachment of one or more radioactive isotopes prior to administration to a subject.

In still further embodiments, diagnostic and/or treatment methods are provided, these methods comprising the administration of the carrier molecule to a subject. In the case of treatment methods, one or more therapeutic agents are attached to the carrier molecule. In diagnostic methods, one or more detectable moieties are attached to the carrier molecule. In additional embodiments, a combined diagnostic and treatment method is provided wherein one or more therapeutic agents and one or more detectable moieties are both attached to the carrier molecule such that the carrier molecule can be used for both diagnostic methods and treatment. In still further embodiments, the therapeutic agent and the detectable diagnostic moiety are the same compound or material—i.e., the attached moiety is not only detectable but also therapeutic (e.g., gallium-68). Other embodiments provide ex vivo diagnostic methods wherein a bodily fluid or tissue sample is collected from a subject and then contacted with a carrier molecule having one or more detectable moieties attached thereto.

As used herein, the term “diagnosing” includes determining the presence or absence of a disorder, determining the likelihood that a particular disorder will develop in the future, and/or determining the status of a previously confirmed disorder in a subject. For example, in the case of cancer, the term diagnosing encompasses determining the presence or absence of cancer, the stage of cancer, and/or the detection of the presence, absence, or stage of a precancerous condition in a patient. Determining the status of a previously confirmed disorder also includes determining the progress, lack of progress, decline or remission of the disorder (e.g., a macrophage-related disorder). And the term “treatment” (as well as “treating”) is intended to mean the broadest definition, including not only curing or eliminating a disorder (e.g., a disease or medical condition), but also reducing, slowing the progress of, or ameliorating one or more effects of the disorder.

Macrophage-related and other CD206 expressing cell-related disorders for which the compositions and methods herein may be used include, but are not limited to: acquired immune deficiency syndrome (AIDS), acute disseminated encephalomyelitis (ADEM), Addison's disease, agammaglobulinemia, allergic diseases, alopecia greata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behçet's disease, Berger's disease, Bickerstaff's encephalitis, Blau syndrome, bullous pemphigoid, cardiovascular vulnerable plaque, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barré syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjögren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, systemic rheumatic diseases, Takayasu's arteritis, temporal arteritis (aka “giant cell arteritis”), thin-capped fibro-atheroma, thrombocytopenia, Tolosa-Hunt syndrome, transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Applicants have discovered that tilmanocept as well as other related carrier molecules described in the '990 patent, as well as other carrier molecules based on a dextran backbone, bind exclusively to the mannose receptor protein CD206 found on the surface of macrophages and certain other cells (e.g., Kaposi's sarcoma spindle cells and dendritic cells) when administered to mammals or when contacted with CD206 expressing cells ex vivo. No other receptors are believed to specifically bind or transduce these carrier molecules, even though there are numerous other carbohydrate-binding receptors found in mammals. CD206 is a C-type lectin protein found on the surface of macrophages and certain other types of cells. The finding that the CD206 protein, found for example on the surface of macrophages, appears to be the sole gateway for tilmanocept binding in mammalian patients means that the tilmanocept carrier molecule (as well as related carrier molecules) can be used as the basis for preparing a variety of therapeutically and diagnostically effective molecular species for use in the diagnosis and/or treatment of macrophage related disorders and other CD206 expressing cell-related disorders.

The carrier molecules used in the compositions, kits and therapeutic and diagnostic methods described herein are used to deliver a detectable moiety and/or a therapeutic agent (e.g., a cytotoxic agent) to cells. These carrier molecules include one or more features which allow a detectable moiety and/or a therapeutic agent to be attached to the carrier molecule, as well as one or more receptor ligands (also referred to as receptor substrates) which direct the carrier molecules to bind exclusively to CD206. In this manner, the detectable moiety or therapeutic agent is delivered to cells expressing CD206 for purposes of subsequent detection (i.e., for diagnostic purposes) and/or for therapeutic purposes (e.g., to target a cytotoxic agent to cells expressing CD206, or neighboring cells to CD206-expressing cells).

It has also been discovered that the carrier molecules described herein not only bind to CD206 on the cell surface, but are also internalized into the cell. Once inside macrophages, the carrier molecules persist in what appears to be stable, non-digesting vesicles. This additional finding means that the amount of carrier molecules which bind to cell in vivo is not limited to the number of CD206 receptors on the cell surface, since once a carrier molecule is internalized the CD206 protein to which that carrier molecule attached will be available for binding an additional carrier molecule through recycling. This aspect allows a greater number of carrier molecules and attached detectable and/or therapeutic moieties to bind to (including within) the targeted cells, thus improving diagnostic detection and/or the amount of therapeutic agent delivered to the targeted cells. It should be noted that, unless the context indicates otherwise, wherever reference is made to carrier molecules bound to CD206 expressing cells, this will be understood to include carrier molecules which have attached to CD206 and then internalized into the cell.

In the case of ex vivo diagnostic testing, as further described herein, in some embodiments it may be desirable to prevent or limit the internalization of carrier molecules. The reason for this is that some of the ex vivo diagnostic methods herein are based on correlating the number of carrier molecules bound to cells to the number of macrophages or other CD206 expressing cells. If the carrier molecules are internalized into the cells, more carrier molecules are able to attach to the CD206 receptors, thus making it more difficult in some instances to correlate the number of bound carrier molecules to the number of CD206 expressing cells. As described below, one means of preventing or limiting the internalization of carrier molecules is to reduce the temperature of a mixture of bodily fluid and carrier molecules during an incubation period to below the normal physiological temperature (i.e., normal body temperature) of the mammalian subject. The highest level of inhibition of carrier molecule internalization occurs at temperatures slightly above 0° C. (as discussed below).

The carrier molecules used herein generally comprise a dextran backbone of the type described in the '990 patent. Thus, the backbone comprises a plurality of glucose moieties (i.e., residues) primarily linked by α-1,6 glycosidic bonds. Other linkages such as α-1,4 and/or α-1,3 bonds may also be present. In some embodiments, the dextran backbone has a MW of between about 1 kDa and about 50 kDa, while in other embodiments the dextran backbone has a MW of between about 5 and about 25 kDa. In still other embodiments, the dextran backbone has a MW of between about 8 and about 15 kDa, such as about 10 kDa. While in other embodiments the dextran backbone has a MW of between about 1 and about 5 kDa, such as about 2 kDa. The MW of the dextran backbone may be selected based upon the particular disorder to be diagnosed, evaluated, or treated, as well as whether the macromolecular construct is to be used for treatment, diagnosis, or evaluation.

By way of one example, carrier molecules having smaller MW dextran backbones may be appropriate for instances where the molecule is desired to cross the blood-brain barrier, or when reduced residence time is desired (i.e., the duration of binding to CD206 is reduced). Carrier molecules having larger MW dextran backbones may be appropriate for instances where increased residence time is desired (i.e., the duration of binding to CD206 is increased). In still other embodiments, carrier molecules having smaller MW dextran backbones (e.g., about 1 to about 5 kDa) may be employed when more efficient receptor substrates are attached to the dextran backbone (e.g., branched mannose moieties, as described below). More efficient receptor substrates will bind to CD206 for longer durations and/or more effectively, thus allowing for the use of smaller dextran backbones.

In addition to the dextran backbone, the carrier molecule further includes one or more receptor substrates which bind to CD206, wherein the receptor substrates are conjugated to the dextran backbone. Each receptor substrate attached to the dextran backbone comprises one or more residues selected from the group consisting of mannose, fucose, n-acetylglucosamine, D-galactose, n-acetylgalactoseamine, sialic acid and neuraminic acid, attached to one or more of the glucose residues of the dextran backbone. In some embodiments, receptor substrates are attached to between about 10% and about 50% of the glucose residues of the dextran backbone, or between about 20% and about 45% of the glucose residues, or between about 25% and about 40% of the glucose residues. (It should be noted that the MWs referenced herein, as well as the number and degree of conjugation of receptor substrates, leashes, and diagnostic/therapeutic moieties attached to the dextran backbone refer to average amounts for a given quantity of carrier molecules, since the synthesis techniques will result in some variability.)

In some embodiments each receptor substrate comprises a single residue of mannose, fucose, n-acetylglucosamine, D-galactose, n-acetylgalactoseamine, sialic acid or neuraminic acid attached to a separate glucose residue (i.e., each receptor substrate is a monosaccharide). In other embodiments two or more receptor substrates (which may be the same or different) are conjugated to each other and attached to the dextran backbone at a single glucose residue. Thus, in these embodiments each receptor substrate comprises a disaccharide, oligosaccharide or polysaccharide. In the case of a polysaccharide receptor substrate, one embodiment comprises mannan, in particular branched mannan.

In one particular embodiment, the carrier molecule comprises a dextran backbone (e.g., having a MW of between about 1 and about 50 kDa) to which at least one mannose residue is attached, optionally along with one or more residues of fucose, n-acetylglucosamine, D-galactose, n-acetylgalactoseamine, sialic acid and neuraminic acid. In still further embodiments, one or more branched mannose residues are attached to one glucose moiety of the dextran backbone. A branched mannose residue means a di-, oligo- or polysaccharide comprising a mannose residue to which, individually or in combination, one more mannose, fucose, n-acetylglucosamine, D-galactose, n-acetylgalactoseamine, sialic acid or neuraminic acid residues are attached, either linearly or as one or more branches. For example, some embodiments of the carrier molecule comprise a dextran backbone having at least one receptor substrate attached to a glucose moiety of the dextran, wherein that receptor substrate comprises three or more mannose residues (linear or branched). Such additional mannose residues provide increased binding to CD206, thereby allowing, for example, the use of smaller MW dextran backbones.

The receptor substrates are attached to the glucose moieties of the dextran backbone directly or indirectly. In some embodiments, the receptor substrates are attached via leashes which are first attached to at least some of the glucose residues of the dextran backbone (e.g., leashes are attached to between about 50% and 100% of the glucose moieties, or between about 70% and about 95%, or even between about 80% and 90%). The same leash may be attached at all of the locations, or two or more different leashes may be used.

As described in the '990 patent, in some embodiments a plurality of amino-terminated leashes are attached to the majority of the glucose moieties, wherein the amino-terminated leashes comprise —O(CH₂)₃S(CH₂)₂NH₂ such that a hydroxyl group of the glucose residue of the dextran backbone is replaced by the amino-terminated leash. The leash may be attached to the dextran backbone by allylating at least some of the hydroxyl groups on the dextran backbone using allyl bromide. Then, the allyl groups are reacted with aminoethanethiol hydrochloride to produce a dextran backbone having a plurality of —O(CH₂)₃S(CH₂)₂NH₂ leashes. To provide the CD206 binding, receptor substrates (as described above) are conjugated to the amino group of at least some of the leashes. This may be accomplished by the methods described in the '990 patent, or in other ways known to those skilled in the art. By way of example, mannose and/or galactose is conjugated to the amino group of some of the leashes. As discussed above, the attached receptor substrate may be a single moiety, or a linear or branched chain of two or more receptor substrates.

Various other leashes known to those skilled in the art or subsequently discovered may be used in place of (or in addition to) —O(CH₂)₃S(CH₂)₂NH₂. These include, for example, bifunctional leash groups such as alkylene diamines (H₂N—(CH₂)ᵣ—NH₂), where r is from 2 to 12; aminoalcohols (HO—(CH₂)ᵣ—NH₂), where r is from 2 to 12; aminothiols (HS—(CH₂)ᵣ—NH₂), where r is from 2 to 12; amino acids that are optionally carboxy-protected; ethylene and polyethylene glycols (H—(O—CH₂—CH₂)ₙ—OH, where n is 1-4). Suitable bifunctional diamines include ethylenediamine, 1,3-propanediamine, 1,4-butanediamine, spermidine, 2,4-diaminobutyric acid, lysine, 3,3′-diaminodipropylamine, diaminopropionic acid, N-(2-aminoethyl)-1,3-propanediamine, 2-(4-aminophenyl)ethylamine, and similar compounds. One or more amino acids also can be employed as the bifunctional leash molecule, such as β-alanine, γ-aminobutyric acid or cysteine, or an oligopeptide, such as di- or tri-alanine.

Other bifunctional leashes include, but are not limited to:

—NH—(CH₂)ᵣ—NH—, where r is from 2-5,

—O—(CH₂)ᵣ—NH—, where r is from 2-5,

—NH—CH₂—C(O)—,

—O—CH₂—CH₂—O—CH₂—CH₂—O—,

—NH—NH—C(O)—CH₂—,

—NH—C(CH₃)₂C(O)—,

—S—(CH₂)ᵣC(O), where r is from 1-5,

—S—(CH₂)ᵣNH, where r is from 2-5,

—S—(CH₂)ᵣO, where r is from 1-5,

—S—(CH₂)—CH(NH₂)—C(O)—,

—S—(CH₂)—CH(COOH)—NH—,

—O—CH₂CH(OH)—CH₂—S—CH(CO₂H)—NH—,

—O—CH₂CH(OH)—CH₂—S—CH(NH₂)—C(O)—,

—O—CH₂CH(OH)—CH₂—S—CH₂—CH₂—NH—,

—S—CH₂C(O)—NH—CH₂—CH₂—NH—, and

—NH—O—C(O)—CH₂—CH₂—O—P(O₂H)—.

The macromolecules used in the therapeutic and diagnostic methods and compositions described herein further include a detectable moiety and/or a therapeutic agent which is attached to the carrier molecule. In some embodiments, the detectable moiety and/or a therapeutic agent is attached directly to a glucose residue of the carrier molecule (e.g., via covalent bonding chemistry and synthesis techniques), while in other embodiments the detectable moiety and/or therapeutic agent is attached using one or more leashes (which may be the same or different leashes as those used to attach receptor substrates), as described below.

In still further embodiments, a chelator is attached to the carrier molecule for use in attaching a detectable moiety and/or therapeutic agent. In some embodiments using leashes attached to the carrier backbone, and as described in the '990 patent, a chelator is conjugated to the amino group of some of the leashes and is used to bind the detectable moiety thereto. Suitable chelators include ones known to those skilled in the art or hereafter developed, such as, for example, tetraazacyclododecanetetraacetic acid (DOTA), mercaptoacetylglycylglycyl-glycine (MAG3), diethylenetriamine pentaacetic acid (DTPA), dimercaptosuccinic acid, diphenylethylene diamine, porphyrin, iminodiacetic acid, and ethylenediaminetetraacetic acid (EDTA).

In one particular embodiment, the carrier molecule comprises a dextran backbone of between about 10 and about 15 glucose moieties, or about 11 to about 12 glucose moieties, or about βglucose moieties. Receptor substrates are conjugated to between about 2 and about 4 of the glucose moieties, or in other embodiments two of the glucose moieties. The receptor substrates are attached directly to the glucose moieties or indirectly using leashes (e.g., one of those previously described herein, such as —O(CH₂)₃S(CH₂)₂NH₂). The receptor substrates comprise branched oligosaccharide moieties, each comprising three or more attached moieties chosen from the group consisting of mannose, fucose, n-acetylglucosamine, D-galactose, n-acetylgalactoseamine, sialic acid and neuraminic acid. In some instances, each receptor substrate attached to one of the glucose residues of the dextran backbone comprises a branched oligosaccharide comprising four or more attached moieties chosen from the group consisting of mannose, fucose, n-acetylglucosamine, D-galactose, n-acetylgalactoseamine, sialic acid and neuraminic acid. In further embodiments, each receptor substrate attached to one of the glucose residues of the dextran backbone comprises a branched oligosaccharide comprising five or more, or even six or more attached moieties chosen from the group consisting of mannose, fucose, n-acetylglucosamine, D-galactose, n-acetylgalactoseamine, sialic acid and neuraminic acid. In still further embodiments, each receptor substrate attached to one of the glucose residues of the dextran backbone comprises a branched oligosaccharide comprising four or more, or in some instances five or more, mannose residues. In these embodiments of a carrier molecule comprising a dextran backbone of about 10-15, 11-12 or 13 glucose moieties, a chelator such as DTPA and/or DOTA is conjugated to one or more of the glucose moieties not having a receptor substrate, either directly or via a leash, so as to provide attachment points for a detectable moiety and/or a therapeutic agent.

In other embodiments, the chelator is not needed, particularly when the detectable moiety and/or therapeutic agent can be attached directly to one of the glucose residues of the dextran backbone or to one of the leashes attached to a glucose residue of the dextran backbone. By way of example, amine reactive dyes such as various commercially available fluorophores readily react with the amino group of the leash —O(CH₂)₃S(CH₂)₂NH₂. These dyes typically are in the form of N-hydroxysuccinimide (NHS) esters, and may be reacted with amino groups on carrier molecule leashes simply by mixing the carrier molecule and NHS ester of the dye in a cosolvent (e.g., DMSO or DMF). Thus, for some applications a chelator is not necessary on the carrier molecule.

In one specific embodiment, the carrier molecule comprises tilmanocept (the structure of which was described in the Background section herein). A detectable moiety such as an amine reactive dye can be readily attached to tilmanocept simply by reacting the dye with the amino group on the unconjugated amine side chains (i.e., the leashes which are not bound to a mannose residue or DTPA). A radioactive isotope also can be readily attached to tilmanocept in order to provide a detectable moiety and/or a therapeutic agent.

In one particular embodiment, the carrier molecule is tilmanocept which, as described previously, includes the chelator DTPA attached to the amino group of a portion of the leashes. A radioactive isotope such as ^{99m}Tc is bound to the DTPA shortly before use for diagnostic purposes (i.e., acts as a detectable moiety). By way of specific example, and as described in U.S. Pat. No. 8,545,808 which is incorporated by reference herein, a kit comprising tilmanocept powder in a vial is provided, wherein the vial contains a mixture of 250 mcg tilmanocept, 20 mg trehalose dihydrate, 0.5 mg glycine, 0.5 mg sodium ascorbate, and 0.075 mg stannous chloride dihydrate. The contents of the vial are lyophilized and are under nitrogen. Prior to administration to a subject, a sodium pertechnetate Tc 99m solution is aseptically added to the vial of tilmanocept powder in order to radiolabel the tilmanocept with Tc 99m. Thereafter, a diluent such as sterile saline or a sterile, buffered diluent solution comprising 0.04% (w/v) potassium phosphate, 0.11% (w/v) sodium phosphate (heptahydrate), 0.5% (w/v) sodium chloride, and 0.4% (w/v) phenol, with a pH of about 6.8-7.2, is added to the vial. The resulting radiolabeled tilmanocept is then ready for administration to a patient (e.g., by intravenously). Other carrier molecules described herein may be radiolabeled in a similar manner, with ^{99m}Tc or a variety of other radioactive isotopes. Radioactive therapeutic agents may be similarly attached to the carrier molecules, as desired—either in combination with one or more detectable moieties or other therapeutic agents or alone.

As used herein, the term “detectable moiety” means an atom, isotope, or chemical structure which is: (1) capable of attachment to the carrier molecule; (2) non-toxic to humans or other mammalian subjects; and (3) provides a directly or indirectly detectable signal, particularly a signal which not only can be measured but whose intensity is related (e.g., proportional) to the amount of the detectable moiety. The signal may be detected by any suitable means, including spectroscopic, electrical, optical, magnetic, auditory, radio signal, or palpation detection means.

Suitable detectable moieties include, but are not limited to radioisotopes (radionuclides), fluorophores, chemiluminescent agents, bioluminescent agents, magnetic moieties (including paramagnetic moieties), metals (e.g., for use as contrast agents), RFID moieties, enzymatic reactants, colorimetric release agents, dyes, and particulate-forming agents.

By way of specific example, suitable detectable moieties include, but are not limited to:
contrast agents suitable for magnetic resonance imaging (MRI), such as gadolinium (Gd³⁺), paramagnetic and superparamagnetic materials such as superparamagnetic iron oxide;
contrast agents suitable for computed tomographic (CT) imaging, such as iodinated molecules, ytterbium and dysprosium;
radioisotopes suitable for scintigraphic imaging (or scintigraphy) such as technetium-99m, ^{210/212/213/214}Bi, ^{131/140}Ba, ^{11/14}C, ⁵¹Cr, ^{67/68}Ga, ¹⁵³Gd, ^{88/90/91}Y, ^{123/124/125/131}I, ^{111/115m}In, ¹⁸F, ¹⁰⁵Rh, ¹⁵³Sm, ⁶⁷Cu, ¹⁶⁶Ho, ¹⁷⁷Lu, ¹⁸⁶Re and ¹⁸⁸Re, ^{32/33}P, ^{46/47}Sc, ^{72/75}Se, ³⁵S, ¹⁸²Ta, ^{123m/127/129/132}Te, ⁶⁵Zn and ^{89/95}Zr;
gamma-emitting agents suitable for single-photon emission computed tomography (SPECT), such as ^{99m}Tc, ¹¹¹In, ^{117m}Sn and ¹²³I;
dyes and fluorescent agents suitable for optical imaging, including but not limited to, dyes such as cyanine fluorophores (e.g., Cy3. Cy5, Cy5.5, Cy7), Alexa Fluor® dyes (available from Molecular Probes, Inc.) anthracene, coumarin, fluorescein, rhodamine, pHrodo™, green fluorescence protein, biarsenical-tetracysteine, 2-(4)-dehydroxycoelenterazine, 5-FAM-diacetate, isocyanine green, and deriviatives thereof; and
agents suitable for positron emission tomography (PET) such as ¹⁸F.

In one particular embodiment, the carrier molecules used in the therapeutic and diagnostic methods and compositions described herein include the cyanine dye Cy3. Cy3-tilmanocept can be prepared, for example, by treating a dimethylsulfoxide (DMSO) solution of mannosyl-dextran prepared using methods described in Vera et al JNM 2001, 42:951-9, dropwise with a DMSO solution of the N-hydroxy succinimide ester of Cy3. After standing at room temperature for 1 hour, the reaction mixture was purified to provide Cy3-tilmanocept.

In another particular embodiment, the fluorescent agent Alexa Fluor® 488 (Alexa Fluor® 488 carboxylic acid, succinimidyl ester) is attached to the carrier molecule in a manner similar to Cy3.

In some embodiments, the carrier molecules used in the therapeutic and diagnostic methods and compositions described herein include a therapeutic agent which is attached to the carrier molecule—either in place of a detectable moiety or in conjunction therewith. As used herein, the term “therapeutic agent” means an atom, isotope, or chemical structure which is effective in curing or eliminating a disease or other condition, as well those which are effective in reducing, slowing the progress of, or ameliorating the adverse effects of a disease or other condition.

In some embodiments, the therapeutic agent comprises a high energy killing isotope which has the ability to kill macrophages and tissue in the surrounding macrophage environment. Suitable radioisotopes include: ^{210/212/213/214}Bi, ^{131/140}Ba, _{11/14}C, ⁵¹Cr, ^{67/68}Ga, ¹⁵³Gd, ^{99m}Tc, ^{88/90/91}Y, ^{123/124/125/131}I, ^{111/115m}In, ¹⁸F, ¹⁰⁵Rh, ¹⁵³Sm, ⁶⁷Cu, ¹⁶⁶Ho, ¹⁷⁷Lu, ¹⁸⁶Re and ¹⁸⁸Re, ^{32/33}P, ^{46/47}Sc, ^{72/75}Se, ³⁵S, ¹⁸²Ta, ^{123m/127/129/132}Te, ⁶⁵Zn, ^{89/95}Zr.

In other embodiments, the therapeutic agent comprises a non-radioactive species selected from, but not limited to, the group consisting of: Bi, Ba, Mg, Ni, Au, Ag, V, Co, Pt, W, Ti, Al, Si, Os, Sn, Br, Mn, Mo, Li, Sb, F, Cr, Ga, Gd, I, Rh, Cu, Fe, P, Se, S, Zn and Zr.

In still further embodiments, the therapeutic agent is selected from the group consisting of cytostatic agents, alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radiosensitizing drugs. By way of more specific example, the therapeutic agent is selected from the group consisting of mechlorethamine, triethylenephosphoramide, cyclophosphamide, ifosfamide, chlorambucil, busulfan, melphalan, triaziquone, nitrosourea compounds, adriamycin, caminomycin, daunorubicin (daunomycin), doxorubicin, isoniazid, indomethacin, gallium(III), ⁶⁸gallium(III), aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, taxol, taxane, cytochalasin B, gramicidin D, ethidium bromide, emetine, tenoposide, colchicin, dihydroxy anthracin dione, mitoxantrone, procaine, tetracaine, lidocaine, propranolol, puromycin, ricin subunit A, abrin, diptheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, esperamicins, and dynemicins.

In embodiments wherein the therapeutic agent is a hormone or hormone antagonist, the therapeutic agent may be selected from the group consisting of prednisone, hydroxyprogesterone, medroprogesterone, diethylstilbestrol, tamoxifen, testosterone, and aminogluthetimide.

In embodiments wherein the therapeutic agent is a prodrug, the therapeutic agent may be selected from the group consisting of phosphate-containing prodrugs, thiophosphate-containing prodrugs, sulfate containing prodrugs, peptide containing prodrugs, (-lactam-containing prodrugs, optionally substituted phenoxyacetamide-containing prodrugs, optionally substituted phenylacetamide-containing prodrugs, 5-fluorocytosinem, and 5-fluorouridine prodrugs that can be converted to the more active cytotoxic free drug.

The therapeutic agent is attached to the carrier molecule in a variety of ways. In some embodiments, and as described in the '990 patent, a chelator is conjugated to the amino group of some of the leashes and is used to bind the therapeutic agent thereto. Suitable chelators include ones known to those skilled in the art or hereafter developed, such as, for example, tetraazacyclododecanetetraacetic acid (DOTA), mercaptoacetylglycylglycyl-glycine (MAG3), diethylenetriamine pentaacetic acid (DTPA), dimercaptosuccinic acid, diphenylethylene diamine, porphyrin, iminodiacetic acid, and ethylenediaminetetraacetic acid (EDTA).

The macromolecular compounds described herein may be administered in a variety of ways, using any of a variety of pharmaceutically acceptable carriers and vehicles. For example, a pharmaceutical preparation comprising the carrier molecule having one or more detectable moieties and/or therapeutic agents attached thereto, in combination with a pharmaceutically acceptable carrier is administered via intravenous injection, subcutaneous injection, intradermal injection, parenchymal introduction, inhalation, pulmonary lavage, suppository, or oral, sublingual, intracranial, intraocular, intranasal, or intraaural introduction. The diagnostic methods of the present invention include not only detecting the presence of absence of a disorder, but also tracking the progress of treatment for a disorder such as by detecting CD206 expressing cells at a predetermined target location at a first time, administering treatment (by the treatment methods described herein or other treatment methods), and detecting CD206 expressing cells at a predetermined target location at a later second time. A difference in CD206 expressing cells, if sufficiently significant, can be used to demonstrate the efficacy or lack of efficacy of the treatment. Diagnosing also includes identifying subjects predisposed to a disorder or to diagnose markers indicating a disorder is likely to become symptomatic or develop in the future.

In addition to the in vivo methods of diagnosing and treating various disorders, the carrier molecules described above, particularly when one or more detectable moieties are attached to the carrier molecule, can be used in ex vivo diagnostic methods and diagnostic kits. These methods and kits are used to quantitate the number of cells expressing CD206 in a bodily fluid sample, which is then used for diagnostic purposes. For example, the determined number of cells expressing CD206 in a given quantity of bodily fluid is used to diagnose the presence or absence of a medical condition, or is used to determine the status of a previously confirmed medical condition in a patient by comparing the number of CD206 expressing cells to previously acquired or compiled data.

In one specific example, these ex vivo diagnostic methods and kits are used to diagnose the presence of rheumatoid arthritis (“RA”) in a mammalian subject and to assess the stage or treatment progress of rheumatoid arthritis in a mammalian subject previously determined to have RA. In the case of RA, the bodily fluid collected from the subject is synovial fluid, extracted from a joint which is suspected or known to be affected by RA.

The bodily fluid is contacted with the carrier molecule having at least one detectable moiety attached thereto such that the carrier molecule binds to cells expressing CD206 which are present in the bodily fluid. This contacting step may be accomplished in any suitable container such as a suitably seized vial which may be capped to allow thorough mixing of the fluid and the carrier molecule. In one embodiment, the fluid and carrier molecule are combined in a centrifuge vial (also known as a centrifuge tube). Following mixing of the fluid and carrier molecule, the resulting mixture is incubated for a predetermined period of time sufficient to allow the carrier molecule to bind to CD206 on the surface of cells in the bodily fluid.

In some embodiments, incubation is performed at a temperature below the subject's physiological temperature in order to inhibit the carrier molecule from being internalized into the cells. If carrier molecules are internalized into cells, the CD206 receptors to which the molecules attached become available once again for attachment of additional carrier molecules. However, by reducing the incubation temperature the internalization of carrier molecules is inhibited or prevented. In some embodiments, the mixture is incubated at a temperature of between about 0° C. and about 25° C.; in other embodiments between about 1° C. and about 10° C.; and in still further embodiments between about 1° C. and about 4° C. In one particular embodiment, the mixture is incubated at a temperature of about 4° C.

In some embodiments, the mixture is incubated for a duration of between about 1 minute to about 1 day. In some embodiments, the mixture is incubated for a duration of between about 1 minute to about 1 hour. In other embodiments, the mixture is incubated for a duration of between about 1 minute to about 5 minutes.

Following incubation, the cells of the bodily fluid are separated from unbound carrier molecules. Since the cells are insoluble and the carrier molecules are water soluble, separation can be accomplished by centrifugation. The unbound carrier molecules will remain in the liquid phase, and thus may be easily removed (e.g., by decantation or using a pipette). Thereafter, the level of the detectable moiety in the cell portion (i.e., the solid phase following centrifugation) is measured. The measurement method will depend upon the nature of the detectable moiety.

By way of example, when the detectable moiety is a dye such as a fluorophore, measuring the level of detectable moiety in the cell fraction comprises spectroscopically measuring the level of fluorescence of the cell fraction.

Embodiments of the present invention further include a diagnostic kit for quantitating the number of cells expressing CD206 in a bodily fluid sample, which is then used for diagnostic purposes. The kit generally comprises:
(a) a first sealed container containing a carrier molecule as described previously herein, with at least one spectroscopically detectable moiety attached thereto (e.g. a fluorophore);
(b) a second sealed container containing a diluent;
(c) at least one centrifuge vial; and
(d) at least one cuvette for use in the spectroscopic measuring device.

The next sections provide examples demonstrating carrier molecule binding to CD206 as well as describe various CD206 expressing cell-related disorders which may be diagnosed and/or treated with the carrier molecules described herein (including data and diagnostic/treatment methods). It will be understood, however, that the specific carrier molecules described in the following examples are merely exemplary of those which may be used in diagnosing or treating the disorders discussed below. Thus, any of the previously described carrier molecules may be used in place of those in the specific examples below. In addition, it will also be understood that the present invention is not limited to the diagnosis and/or treatment of the specific disorders discussed below, as these are intended to be merely exemplary of particular embodiments.

### Tilmanocept-Cy3 Binding to Human Macrophages

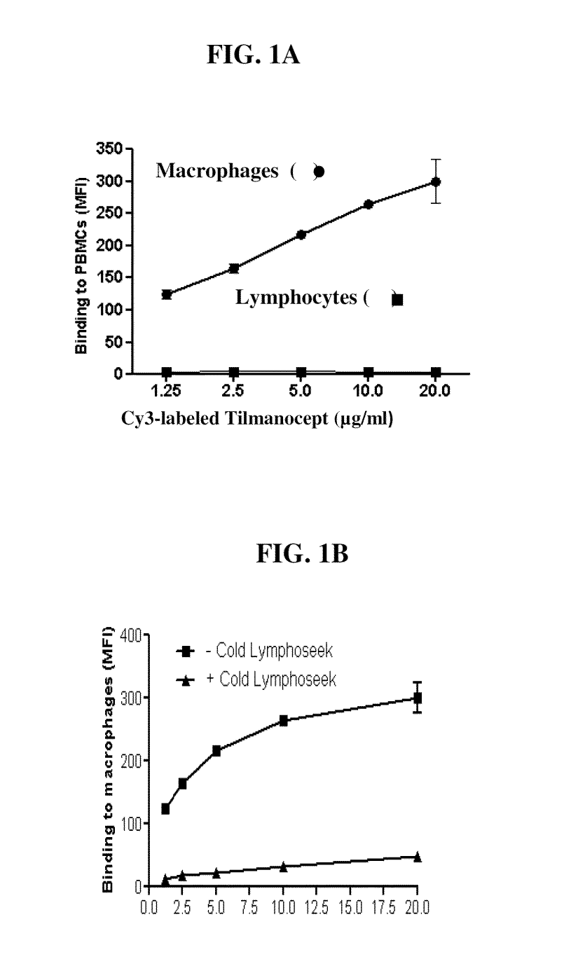

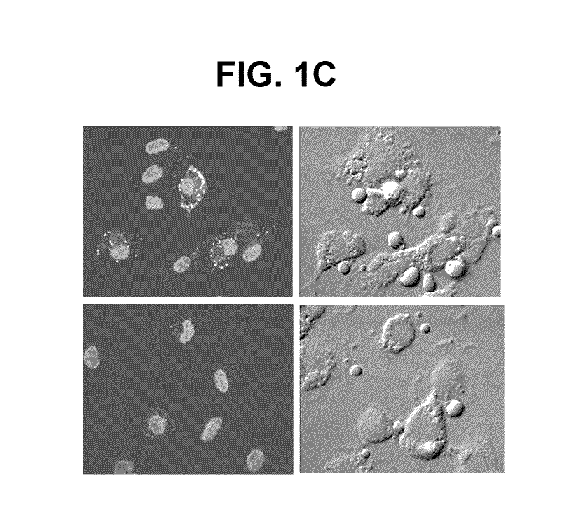

Whether tilmanocept binds to lymphocytes or macrophages was determined using human peripheral blood mononuclear cells (PBMCs). A quantity of PBMCs consisting of lymphocytes and macrophages was cultured for 5 days to enable blood monocytes to differentiate into macrophages (human monocyte-derived macrophages, or “MDMs”), and then pre-treated with or without unlabeled (cold) tilmanocept. Next, the cells were incubated with varying concentrations (1.25, 2.5, 5.0, 10 and 20 μg/mL) of Cy3-labeled tilmanocept (Cy3-tilmanocept). Tilmanocept binding to PBMC cell populations was analyzed by flow cytometry by gating separately for macrophages and lymphocytes. The resulting data showed that tilmanocept binds specifically to the macrophage population in a dose-dependent manner, as shown in FIG. 1A. FIG. 1A depicts fluorescence-activated cell sorting (“FACS”) analysis of PBMCs, focusing on macrophages and lymphocytes. For the macrophages that were pre-treated with cold tilmanocept (100-fold excess), the binding of Cy3-tilmanocept was nearly abolished even at the highest concentrations, as shown in FIG. 1B (FACS analysis showing inhibition of Tilmanocept-Cy3 binding to macrophages in presence of unlabeled Tilmanocept**P<0.005).

To corroborate these findings, MDMs were treated in monolayer culture in a similar way, and fluorescence confocal microscopy experiments were performed. The binding of Cy3-tilmanocept to macrophages was readily apparent and this binding was nearly abolished for macrophages that were pre-treated with cold tilmanocept, as seen in FIG. 1C. Depicted data is representative of two independent experiments, each performed in duplicate, and the results were consistent with receptor-mediated binding of tilmanocept to macrophages. The upper and lower left images in FIG. 1C depict confocal microscopy representative images (magnification: 120×) which show binding (upper left) and inhibition of binding (lower left) of tilmanocept-Cy3 to macrophages in the absence or presence of tilmanocept with no fluorophore, respectively. The gray regions indicate macrophage nuclei, and the white portions indicate tilmanocept-Cy3. The upper and lower right images in FIG. 1C are DIC images which show the individual cell structure of the adjacent fluorescent images (to the left of each DIC image). “DIC” is Differential Interference Contrast (phase contrast microscopy).

Co-Localization of Tilmanocept with the CD206 on Human Macrophages

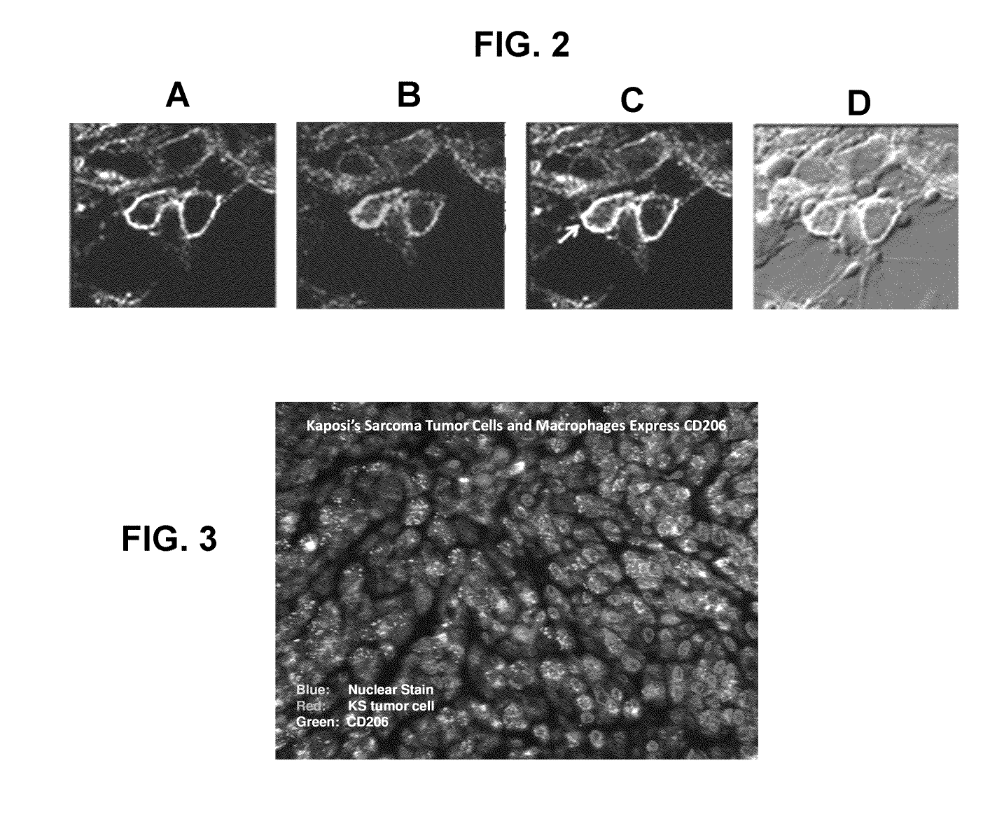

MDM monolayers were incubated with Cy3-tilmanocept for 10 minutes, fixed with paraformaldehyde, incubated with anti-MR primary Ab, and stained with Alexa Fluor 488-conjugated secondary Ab. The monolayers were then analyzed by confocal microscopy. FIG. 2 illustrates representative confocal images (magnification: 160×) showing expression of CD206 (FIG. 2A), tilmanocept binding by the macrophage (FIG. 2B), and co-localization between CD206 and tilmanocept in both confocal and phase contrast images (FIGS. 2C and 2D). The results shown are representative of three independent experiments.

Macrophages are known to be associated with several disease states, such as Kaposi's sarcoma (KS), rheumatoid arthritis (RA) and tuberculosis (TB), wherein macrophages with high CD206 expression localize to disease lesions and can be targeted for imaging using CD206 biomarker technology.

### Diagnosis and Treatment of Kaposi's Sarcoma

Inflammation is a necessary response to numerous disease states, including tumor expression. A major component of this inflammatory process is now recognized to be driven by macrophages, which impact tumor initiation, promotion and progression. For cancer tissues, tumor-associated macrophages (TAMs) have been identified that play important roles in tumor invasion, cancer cell proliferation and metastasis. These M2-type macrophages typically express high levels of CD206. A model tumor for macrophage-dependent progression is Kaposi's sarcoma (KS), as KS is driven by TAMS. There is also strong evidence that KS metastasis is associated with tumor cells that co-express macrophage markers. Thus, macrophages are potentially an important target to exploit in KS pathogenesis.

HIV-associated KS is an aggressive, multi-focal, neoplasm associated with herpes virus (HHV8/KSHV) infection. KS involves cutaneous and visceral tissues, with later disease associated with organ involvement. KS is a form of cancer where inflammation appears to play a critical role in tumor development. KS tumor cells co-expressing various macrophage markers are becoming resistant to current anti-viral approaches for treatment of KS and AIDS. Applicants have discovered that, as tilmanocept and related carrier molecules described above bind to CD206, in the tumor parenchyma where this CD206 expression may be critical pathway in the development of a new antitumor agents directed against TAMs and metastatic tumor cells and tracking their metastatic pattern, diagnosis, and response to therapy.

KS macrophages may be a significant HIV reservoir of infected cells resistant to standard anti-retroviral therapy. The tumor associated forms may directly contribute to KS pathogenesis, although all forms of HIV within tissues in AIDS patients with advanced disease are macrophage tropic.

Liposomal doxorubicin (Doxil® (doxorubicin HCl liposome injection), Janssen Products, LP) is most effective for treating KS resistant to antiretroviral therapy (ART), however it is generally unavailable. Treatments would benefit from a better understanding of the immune makeup of Kaposi sarcoma especially important for monitoring therapeutic responses in general.

Historically, there has been no imaging platform that has been able to identify KS specific lesions or metastatic foci in patients with KS. This has been problematic in delivery of clinical care as physicians are unable to appropriately stage patients with KS, other than the tracking of skin lesions. KS is known to involve lymph nodes and organs, but to date no approach has been able to confirm tumor involvement beyond skin.

In one embodiment, the carrier molecules described above having receptor substrates which bind to CD206 are used to provide methods for effective imaging of KS involved nodes and other visceral sites of disease. In another embodiment, the compositions of the present invention provide methods of defining tumor burden allowing for earlier tumor specific treatment beyond the current use of anti-retroviral therapy alone, which is proving ineffective in growing numbers of KS patients worldwide. In another embodiment, the compositions of the present invention provide methods of tracking tumor metastatic patterns by one of several external imaging methods, including but not limited to scintigraphy, SPECT, SPECT/CT, gamma probing (in vivo or ex vivo), external (ex vivo) or internal (in vivo) fluorescence. In another embodiment, the compositions of the present invention provide methods for tracking response to tumor therapy as indicated in the immediate previous methods or in vitro utilizing biopsy tissue and the same diagnostic agents employed in the laboratory setting.

An elegant precision diagnostic approach to the above is macrophage-targeted imaging mediated via a key receptor, CD206. CD206 has been successfully exploited as the target for precision imaging using tilmanocept, which binds to CD206 by interaction of mannose moieties on the tilmanocept molecule and is taken into the macrophage where it persists in stable non-digesting vesicles. Detectable moieties such as Cy3 or Tc99m allow targeted imaging. This precision targeting mechanism provides a novel pathway to image key functions of the macrophage-driven disease process such as in KS and other macrophage-mediated diseases and disorders. Presence of CD206 allows the compositions of present invention to be used as tumor specific imaging agents capable of identifying both tumor cells as well as TAMs in patients with KS.

In the studies outlined below, a CD206-targeted tilmanocept platform imaging approach was evaluated in Kaposi's sarcoma (KS) derived from AIDS patients. These studies demonstrate that the majority of both TAMS and KS cells express the macrophage marker CD206 that can be specifically targeted with the carrier molecules described herein, such as tilmanocept. This allows, for example, detectable moieties to be targeted to KS lesions for diagnostic purposes. This also provides treatment compositions and methods using the carrier molecules described herein. Applicants tested a large collection of both skin and visceral forms of KS to determine whether CD206 would be present on both KS tumor cells and TAMs. Applicants tested the frequency of macrophage antigens on HHV8/KSHV infected KS tumor cells and the frequency of CD206+ tilmanocept binding cells within KS lesion cell subpopulations.

### Over 96% of KS Lesion Cells Express the Human Mannose Receptor (MR, CD206)

Immunophenotypic analysis of KS lesion cells confirmed that over 96% of both tumor associated macrophages (TAMs) and KS cells express CD206 that can be specifically targeted with the carrier molecules described herein to define the KS lesion or provide targeted treatment of KS. A tissue microscopic array (TMA) containing 66 cases of AIDS KS and controls was obtained from the AIDS and Cancer Specimen Resource (ACSR). MO antigens were identified by IHC studies and results were standardized to the proportion of KSHV LANA+ cells (KS tumor specific marker). The TMA was stained for the presence of HHV8/KSHV latent antigen (LANA), and macrophage markers MAC387 (M1), CD163 (M2), CD68 (pan macrophage), and CD206 (macrophage mannose receptor, M2) to test for prevalence of these antigens in cases of KS. Included in the TMA were skin as well as visceral lesions. The results of the immuno-histochemistry analysis of the 66 cases of KS are shown in Table 1.

TABLE 1

| | MAC387 | CD163 | CD68 | CD206 |
|---|---|---|---|---|
| Staining | (n = 66) | (n = 66) | (n = 61) | (n = 61) |
| Negative | 6.0% | 15.2% | <1% | <1% |
| Macrophage | 19.6% | 12.1% | 9.8% | 3.8% |
| only | | | | |
| Macrophage and | 74.2% | 72.7% | 90.2% | 95.5% |
| KS Tumor Cells | | | | |

Mac387, CD163 and CD68 are macrophage specific markers

Table 1 summarizes the proportion of KS cases expressing macrophage antigens on TAMs and HHV8/KSHV LANA+ tumor cells. The immuno-histochemistry analysis shows that macrophage antigens are highly associated within KS tumor associated cells. The frequency of the CD68 macrophage antigen staining within KS lesions was highly consistent with KS being a tumor with extensive TAM infiltration. Also, as had been reported in a limited number of cases, this extensive analysis confirmed that KS spindle cells also co expressed macrophage antigens including CD206.

Most TAMs in KS tissues were identified with the M2 specific anti-CD163 antibody whereas the M1 anti MAC387 antibody identified a smaller subset of cells. The CD68 antibody also identified a large number of TAMs in more than 90% of tumors. KS tumor spindle cells in general expressed macrophage antigens; however the most prevalent antigen for both KS tumor cells (LANA+) and TAMs was CD206 molecule. Expression of MO antigens and CD206 in relation to level of LANA within tumor tissues was similar across all tissue forms of KS (plaque, oral, visceral). A pilot study of KS tissues from Africa showed the similar results. Most of LANA+ KS tumor cells co-expressed CD206. CD68+ tissue macrophages were also associated with CD206 antigen in African KS tissues. The results confirmed that both TAMs and KS tumor cells express the CD206 macrophage mannose receptor (Uccini et al. AJP March 1997, 150: 929 938).

FIG. 3 depicts a photomicrograph of KS tumor cells showing markers for nuclei (blue), KS tumor cells (red) and CD206 (green), demonstrating the pan-cellular expression of the CD206 human mannose receptor that binds to the carrier molecules described herein.

KS Tumor Cells and Macrophages that Express CD206 Bind and Internalize Tilmanocept-Cy3

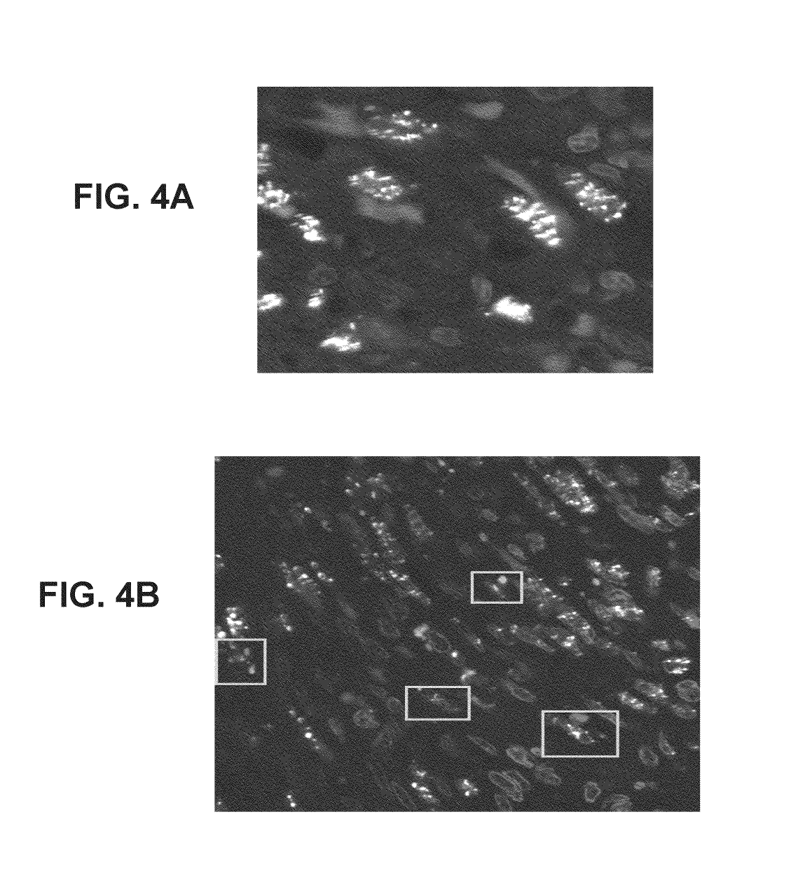

As seen in FIG. 4, both KS tumor cells and macrophages express CD206 and bind tilmanocept-Cy3 (red) on the surface (FIG. 4A) and subsequently internalize tilmanocept-Cy3 into cytoplasmic vesicles (FIG. 4B). Internalization is anticipated to provide for stable accumulation of tilmanocept-Cy3 and potential specific KS lesion imaging. Tilmanocept and the other carriers described herein are thus useful diagnostic and treatment compositions in patients with KS to, for example, stage and quantitatively image tumor specific response to therapy. By extension, other classes of tumors may contain similar, hybrid-like cells and may be imaged with tilmanocept-based agents and clinically addressed with macrophage-targeted therapy.

### Immunofluorescence Stain and Confocal Microscopy

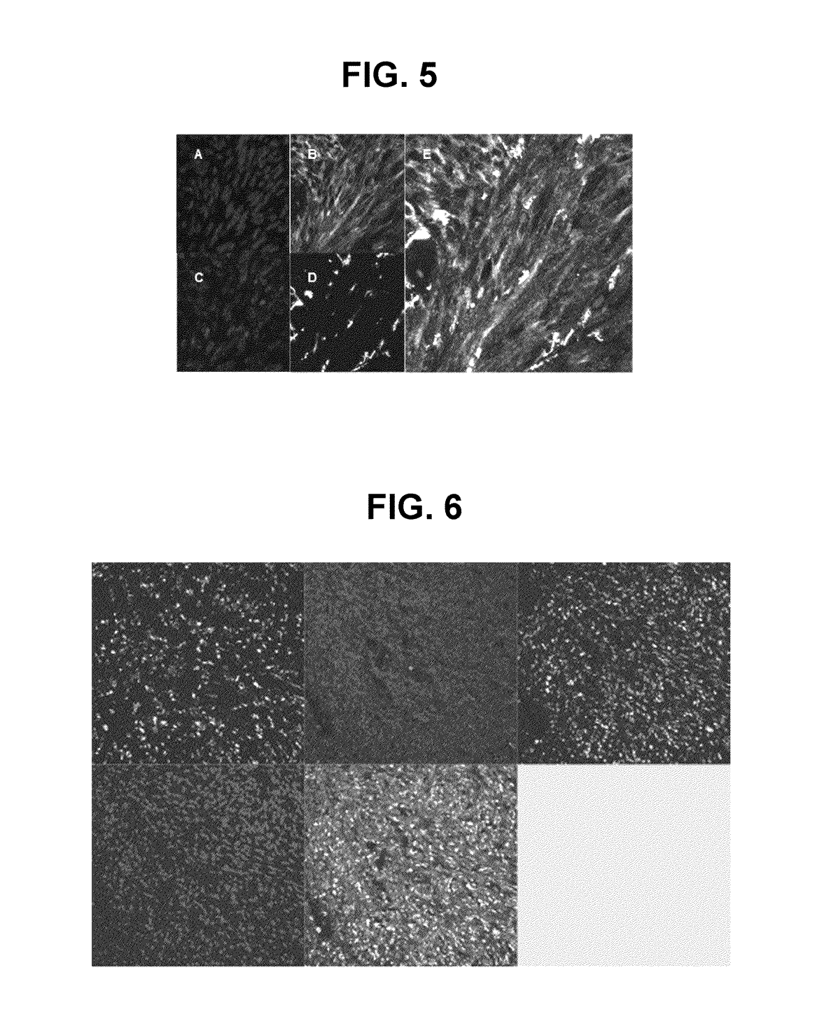

Immunofluorescence stain and confocal microscopy studies determined rates of co-expression of CD206 on both tissue macrophages and LANA expressing KS tumor cells. The immunofluorescence stain and confocal microscopy studies were performed on a tissue microscopic array (TMA) containing 66 cases of AIDS KS and controls obtained from the AIDS and Cancer Specimen Resource (ACSR). The results are shown in FIG. 5 which depicts confocal microscopy representative images showing co-localization of macrophage mannose receptor CD206 on both LANA expressing tumor cells and tissue macrophage. A, DAPI (blue); B, CD206 (green); C, LANA (red); D, CD68 (yellow); E, All merged (63×). Cy3-tilmanocept uptake by HHV8+ KS tumor cells was also examined, and FIG. 6 depicts example confocal images of KS biopsy tissue culture with Cy3 tilmanocept. Confocal images of HHV8+ KS tumor cell biopsy. 25× (CD68, Yellow; Cy3-tilmanocept, Red; HHV8, Green; DAPI, Blue) Cy3-tilmanocept uptake into CD206-expressingmacrophages was also examined.

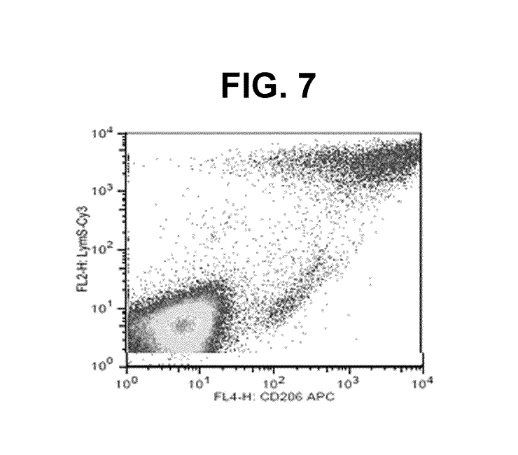

3-day CD206+ macrophage cultures were incubated with Cy3-tilmanocept (100 μg/mL) for 4, 24 and 48 hours at 37° C. Background levels of Cy3 fluorescence were determined in cultures exposed to conjugates at room temperature for the same time periods. Flow cytometric evaluation of Cy3 and CD206 was performed at all time points indicating Cy3-tilmanocept uptake into CD206+ macrophages. FIG. 7 shows a flow cytometric evaluation of Cy3 and CD206 in 3 day CD206+ macrophage cultures incubated with Cy3-tilmanocept.

In light of the above, in further specific embodiments the carrier molecules described herein are used for diagnosing and/or treating KS (and similar types of cancers and tumors). For diagnostic purposes, a detectable moiety such as ^{99m}Tc or ⁶⁸Ga is attached to the carrier molecule (e.g. to a DTPA or DOTA chelator), and the radiolabeled composition administered to a subject such as by subcutaneous or intradermal injection proximal to (i.e., adjacent) the tumor or suspected lesion, intra-tumorally/intra-lesionally injected directly into the tumor or lesion, or by intravenous injection. It will be understood that other detectable moieties described herein, known to those skilled in the art, or hereafter developed may be attached to the carrier molecule for use in diagnosing KS, such as any of a variety of fluorophores. Following administration to a patient, the tumor or lesion site (or suspected tumor or lesion site) is imaged, such as by scintigraphy (e.g., using a gamma camera), single-photon emission computed tomography (SPECT), positron emission tomography (PET), or optical imaging (e.g., when the detectable moiety is a fluorescent dye such as cyanimine). It will be understood, however, that other diagnostic moieties other than those mentioned above may be employed, as well as various other imaging or diagnostic methods for detecting the presence of the labeled carrier molecules in the KS tumor or lesion.

In one specific embodiment for KS diagnostic imaging, the carrier molecule is tilmanocept: dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes. In this particular embodiment, the detectable moiety is ^{99m}Tc or ⁶⁸Ga, and the detectable moiety is attached to a DTPA chelator just prior to use by mixing the carrier molecule with the elute from a ^{99m}Tc generator or a gallium-68 generator, as known to those skilled in the art. In other embodiments, the detectable moiety is Cy-3 and is attached to a leash of tilmanocept, as known to those skilled in the art. For diagnostic imaging of KS using ^{99m}Tc-tilmanocept or ⁶⁸Ga-tilmanocept, in some embodiments the radiolabeled carrier molecule has sufficient radioisotope to provide a dose, when administered locally (e.g., subcutaneuously) to a subject, of between about 0.3 to about 5.0 millicuries, or about 0.5 to about 2.0 millicuries, or about 0.5 or about 1 millicurrie. In other embodiments, such as for diagnostic imaging of KS using ^{99m}Tc-tilmanocept or ⁶⁸Ga-tilmanocept, the radiolabeled carrier molecule has sufficient radioisotope to provide a dose, when administered systemically (e.g., intravenously) to a subject, of between about 2 mCi to about 30 mCi, from about 5 mCi to about 30 mCi, and from about 10 mCi to about 25 mCi. When administered to a subject by injection, the radiolabeled carrier is, in some embodiments, combined with a pharmaceutically acceptable carrier containing one or more excipients, diluents and the like (e.g., sterile saline). For diagnostic imaging of KS using tilmanocept having one or more detectable moieties attached thereto, between about 50 and about 500 micrograms of tilmanocept is administered.

For therapeutic use of the carrier molecules described herein in treating KS, a suitable therapeutic agent is attached to the carrier and the resulting composition is combined with a pharmaceutically acceptable carrier containing one or more excipients, diluents and the like. As with the diagnostic imaging, the carrier molecule with attached therapeutic agent is administered to a patient such as by injection, or even topically to a tumor or lesion. Suitable therapeutic agents for treating KS include functional chemotherapeutic agents such as doxorubicin, daunorubicin, paclitaxel (Taxol®), gemcitabine (Gemzar®), vinorelbine (Navelbine®), bleomycin, vinblastine (Velban®), vincristine (Oncovin®), and etoposide (VP-16). In one particular embodiment, the therapeutic composition comprises Doxorubicin-tilmanocept, which is administered topically (e.g., as a 10 μg dose) or intravenously (e.g., as a 5 mg dose).

### KS Imaging Example 1

Tilmanocept lyophilized powder, marketed by Navidea Biopharmaceuticals Inc. under the name LYMPHOSEEK® Injection kit, is obtained. The tilmanocept powder has a mean diameter of about 7 nm, and is contained in a 0.5 mL vial as a mixture of 0.250 mg tilmanocept, 20 mg trehalose dihydrate, 0.5 mg glycine, 0.5 mg sodium ascorbate and 0.075 mg stannous chloride dihydrate. The tilmanocept powder is then radiolabeled with Tc 99m using sodium 99 mTc-pertechnetate eluted from a Technetium-99m generator. Using a sterile syringe, approximately 92.5 MBq (2.5 mCi) of sodium 99 mTc-pertechnetate in about 0.35 mL is aseptically added to the vial. The vial is gently shaken, and the radiolabeling reaction allowed to proceed at room temperature for at least 10-15 minutes. Normal saline is then added to the vial to bring the contents to 2.5 cc. A buffer is optionally added, as described in U.S. Pat. Pub. No. 2010/0196272 A1, published Aug. 5, 2010, which is incorporated by reference herein.

A single patient dose is 50 mcg of tilmanocept and 0.5 mCi of technetium 99m, as prepared above, totaling 0.5 cc. The radiolabeled tilmanocept is administered by subcutaneous or intradermal injection, within six hours of radiolabeling. In an alternative embodiment, 100 mcg of tilmanocept and 1.0 mCi of technetium, totaling 1 cc, is injected intravenously within six hours of radiolabeling.

Within 30 to 180 minutes of injection, the patient is imaged using Single Photon Emission Computed Tomography (SPECT). The findings of localized radioactivity within the skin lesion(s) is presumptive evidence of mannose binding receptors and/or macrophage activity, which is consistent with the presence of Kaposi's sarcoma, and the absence of such activity would essentially rule out Kaposi's sarcoma.

### KS Imaging Example 2

The following illustrates yet another example of the evaluation of Primary Cutaneous Kaposi's Sarcoma (KS) by SPECT and SPECT/CT Imaging using Lymphoseek® (also known as technetium 99 mTc-tilmanocept injection) a radiopharmaceutical that binds to mannose binding receptors (CD206) that reside on the surfaces of dendritic cells and macrophages. The results indicate that, in patients with primary cutaneous Kaposi's sarcoma, Lymphoseek aids in the detection of Kaposi's sarcoma lesion(s) using single photon emission computed tomography (SPECT) and SPECT computed tomography (SPECT/CT).

An 18 year-old male patient receives a single dose of 50 μg tilmanocept radiolabeled with 2.0 mC±99 mTc by subcutaneous injection. The total volume of 99 mTc-tilmanocept injection is 0.3 to 0.5 mL.

The patient has a marker lesion (>1 cm in diameter) with a confirmed diagnosis of KS (CD 206-expressing cutaneous KS) via punch biopsy. The location of the marker KS lesion is on the extremities: from the shoulder to the metacarpal region or from the groin to the metatarsal region.

The dose is administered by a syringe with a ⅝ inch, 25- or 27-gauge, fixed needle, or other syringe/needle combinations that are acceptable for subcutaneous injections. The injection is made 1.5±0.25 cm distal from the marker lesion, 4-8 cm distal to the marker lesion, or 4-8 cm proximal to the marker lesion.

The patient undergoes a regional dynamic SPECT scan immediately post-injection for a duration of 30 minutes. After the initial scan, the patient undergoes whole body SPECT/CT imaging at 1 hour and whole body SPECT imaging at 4-6 hours post injection. The patient is permitted to leave the imaging center after the 4-6 hour SPECT scan.

Dynamic SPECT/CT (limited CT exposure; GE Infinia Hawkeye 4) imaging occurs immediately following injection for 30 minutes (−3 minutes/rotation). Each dual head spin is segmented into 32)(5.625° angles. SPECT images are acquired at anterior, 45° anterior oblique and lateral positions, with each acquisition being 3-5 minutes in duration for a total of 30-45 minutes.

Acquisition on SPECT/CT systems is performed in a sequential mode. With devices that have a low-dose CT component, data are typically acquired by rotating the X ray detector 220° around the patient, with the X ray tube operated at 140 kV and 2.5 mA. The CT images obtained have an in-plane spatial resolution of 2.5 mm, and of 10 mm in the axial direction. Scan time is approximately 16 seconds per slice, for a total duration of 30-45 minutes for the CT. SPECT/CT systems using a diagnostic CT component are characterized by higher spatial resolution and faster scanning time (approximately 30 seconds for the whole field of view), associated however with higher radiation doses. An attenuation map is created at the end of the CT acquisition time.

### Diagnosis and Treatment of Tuberculosis

Tuberculosis is a respiratory infection caused by the bacteria mycobacterium tuberculosis. In response to TB infection, a patient's immune system forms granulomas which allow TB bacteria to remain within the granulomas for long periods of time with no apparent clinical symptoms of TB. While treatment can reduce the risk of patient developing active TB infection, the granulomas act as a barrier to diagnostic and therapeutic agents. Macrophages are part of the processes of the formation and maintenance of the granulomas. Because of this, Applicants have deduced that the carrier molecules described herein can be used to target CD206 on the surface of the macrophages associated with TB granulomas.

Binding of Tilmanocept to Macrophages Infected with TB

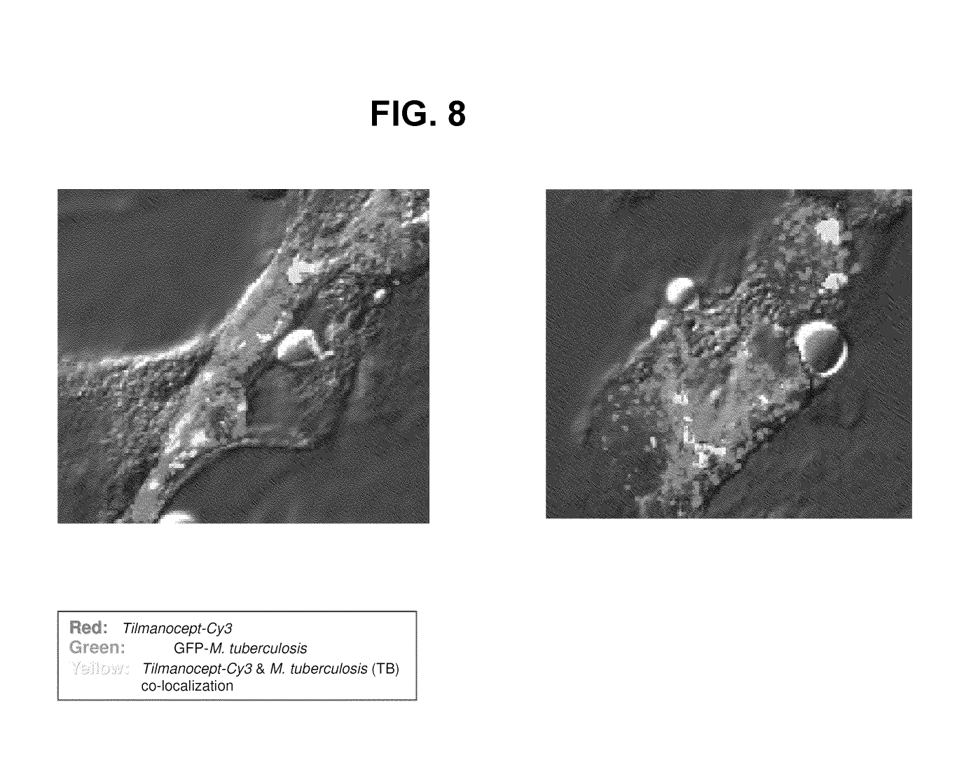

In order to demonstrate the ability of the carrier molecules described herein to bind to macrophages infected with TB and deliver diagnostic and/or therapeutic agents into the interior of the macrophages where the TB bacterium are located, human monocyte-derived macrophages in monolayer culture that make up the components of the TB granulomas were infected with a GFP-expressing M. tuberculosis which was internalized by macrophages (GFP=green fluorescent protein). The infected cells were then exposed to tilmanocept which had been labeled with cyanine (Cy3) dye, and analyzed by confocal microscopy. FIG. 8 depicts the confocal microscopy of the TB-infected macrophages. Red indicates Cy3-tilmanocept, green indicates GFP M. tuberculosis, and yellow indicates the co-localization of Cy3-tilmanocept and GFP M. tuberculosis. Thus, FIG. 8 demonstrates that the Cy3-tilmanocept binds to, and is internalized by the macrophages

In light of the above, in further specific embodiments the carrier molecules described herein are used for diagnosing and/or treating tuberculosis. For diagnostic purposes, a detectable moiety such as ^{99m}Tc or ⁶⁸Ga is attached to the carrier molecule (e.g. to a DTPA or DOTA chelator), and the radiolabeled composition administered to a subject such as by inhalation, intravenous injection or pulmonary lavage. It will be understood that other detectable moieties described herein, known to those skilled in the art, or hereafter developed may be attached to the carrier molecule for use in diagnosing tuberculosis. Following administration to a patient, the subject's lungs are imaged, such as by scintigraphy (e.g., using a gamma camera), single-photon emission computed tomography (SPECT), or positron emission tomography (PET). It will be understood, however, that other diagnostic moieties other than those mentioned above may be employed, as well as various other imaging or diagnostic methods for detecting the presence of the labeled carrier molecules in the subject's lung tissue.

In one specific embodiment for tuberculosis diagnostic imaging, the carrier molecule is tilmanocept: dextran 3-[(2-aminoethyl)thio]propyl 17-carboxy-10,13,16-tris(carboxymethyl)-8-oxo-4-thia-7,10,13,16-tetraazaheptadec-1-yl 3-[[2-[[1-imino-2-(D-mannopyranosylthio)ethyl]amino]ethyl]thio]propyl ether complexes. In this particular embodiment, the detectable moiety is ^{99m}Tc or ⁶⁸Ga, and the detectable moiety is attached to the DTPA chelator just prior to use by mixing the carrier molecule with the elute from a ^{99m}Tc generator or a gallium-68 generator, as known to those skilled in the art. For diagnostic imaging of tuberculosis using ^{99m}Tc-tilmanocept or ⁶⁸Ga-tilmanocept, in some embodiments the radiolabeled carrier molecule has sufficient radioisotope to provide a dose, when administered to a subject, of between about 0.3 to about 5.0 millicuries, or about 0.5 to about 2.0 millicuries, or about 1 millicurrie.

When administered to a subject by inhalation, the radiolabeled carrier is, in some embodiments, combined with a pharmaceutically acceptable vehicle. By way of specific example, the radiolabeled carrier is delivered to the lungs of a human subject by an inhalation device—e.g., a fixed dose inhaler, a dry powder in haler, a metered dose inhaler, or a nebulizer. In one embodiment, the radiolabeled carrier is administered using a metered dose inhaler containing a suspension of the radiolabeled carrier in a vehicle comprising a pharmaceutically acceptable inert liquid propellant such as a chlorofluorocarbon, fluorocarbon or hydrofluoroalkane. By way of more specific example, the metered dose inhaler is configured to deliver about 10 to about 5000 micrograms, or about 10 to about 500 micrograms, of radiolabeled carrier per puff. In still further embodiments, the radiolabeled carrier is suspended in a pharmaceutically acceptable vehicle comprising sterilized water or saline, and administered by nebulization. In yet another embodiment, the radiolabeled carrier is dried to a powder and then administered from a pouch or other container.

For therapeutic use of the carrier molecules described herein in treating TB, a suitable therapeutic agent is attached to the carrier and the resulting composition is combined with a pharmaceutically acceptable vehicle containing one or more excipients, diluents and the like. As with the diagnostic imaging, the carrier molecule with attached therapeutic agent is administered to a patient such as by inhalation, intravenous injection or pulmonary lavage for treating TB (dormant or active infection).

When administered to a subject by inhalation, the therapeutic agent+carrier is, in some embodiments, combined with a pharmaceutically acceptable vehicle. By way of specific example, the therapeutic agent+carrier is delivered to the lungs of a human subject by an inhalation device—e.g., a fixed dose inhaler, a dry powder in haler, a metered dose inhaler, or a nebulizer. In one embodiment, the therapeutic agent+carrier is administered using a metered dose inhaler containing a suspension of the therapeutic agent+carrier in a vehicle comprising a pharmaceutically acceptable inert liquid propellant such as a chlorofluorocarbon, fluorocarbon or hydrofluoroalkane. By way of more specific example, the metered dose inhaler is configured to deliver about 10 to about 5000 micrograms, or about 10 to about 500 micrograms, of therapeutic agent+carrier per puff. In still further embodiments, the therapeutic agent+carrier is suspended in a pharmaceutically acceptable vehicle comprising sterilized water or saline, and administered by nebulization. In yet another embodiment, the therapeutic agent+carrier is dried to a powder and then administered from a pouch or other container. And in yet another embodiment, the therapeutic agent+carrier is suspended in a pharmaceutically acceptable vehicle and administered intravenously, at a dosage of up to 10 mg of the therapeutic agent+carrier molecules.

Suitable therapeutic agents attached to the carrier molecule for treating TB include indomethacin, isoniazid, and/or Ga (optionally as ⁶⁸Ga, such that the composition is used as both a diagnostic and therapeutic composition). In still further embodiments, a composition for both diagnosing and treating tuberculosis is provided wherein both ⁶⁸Ga and Ga (i.e., non-radioactive Ga) are conjugated to the carrier molecule. In other embodiments, two or more of indomethacin, isoniazid and Ga (optionally as ⁶⁸Ga) are conjugated to the carrier molecule. Indomethacin, isoniazid, and Ga are known treatment agents for TB, however, by attaching one or more of these agents to the carrier molecules described herein they are better able to enter the macrophages of granulomas associated with TB wherein the therapeutic agents will target the TB bacterium within those macrophages.

Provided below are examples of compositions and methods which are effective for diagnosing or treating TB.

### Tuberculosis Imaging Example

Tilmanocept lyophilized powder, marketed by Navidea Biopharmaceuticals Inc. under the name LYMPHOSEEK® Injection kit, is obtained. The tilmanocept powder contained in a 0.5 mL vial as a mixture of 0.250 mg tilmanocept, 20 mg trehalose dihydrate, 0.5 mg glycine, 0.5 mg sodium ascorbate and 0.075 mg stannous chloride dihydrate. The tilmanocept powder is then radiolabeled with Tc 99m using sodium ^{99m}Tc-pertechnetate eluted from a Technetium-99m generator. Using a sterile syringe, approximately 92.5 MBq (2.5 mCi) of sodium ^{99m}Tc-pertechnetate in about 0.35 mL is aseptically added to the vial. The vial is gently shaken, and the radiolabeling reaction allowed to proceed at room temperature for at least 10-15 minutes. Normal saline is then added to the vial to bring the contents to 5 cc immediately prior to administration.

A single patient dose of the radiolabeled composition is prepared such that the dose is 100 mcg of ^{99m}Tc-tilmanocept, 1 mCi, totaling 2 cc. The radiolabeled tilmanocept is administered by inhalation within six hours of radiolabeling. The composition is loaded into an aerosol machine and the patient then inhales the composition. Within about 30 to 180 minutes after inhalation, the patients lungs are imaged using Single Photon Emission Computed Tomography (SPECT). The findings of localized radioactivity in the hilar and mediastinal areas of the thoracic cavity will be presumptive evidence of granuloma formation, which is a hallmark of tuberculosis.

### Tuberculosis Treatment Example

Tilmanocept lyophilized powder, marketed by Navidea Biopharmaceuticals Inc. under the name LYMPHOSEEK® Injection kit, is obtained. The tilmanocept powder has a mean diameter of about 7 nm, and is contained in a 0.5 mL vial as a mixture of 0.250 mg tilmanocept, 20 mg trehalose dihydrate, 0.5 mg glycine, 0.5 mg sodium ascorbate and 0.075 mg stannous chloride dihydrate. The tilmanocept powder is then bound to bound to Isoniazid molecules. Normal saline is then added to the vial to bring the contents to 2.5 cc. A buffer is optionally added, as described in U.S. Pat. Pub. No. 2010/0196272 A1, published Aug. 5, 2010, which is incorporated by reference herein.

A single patient dose of the composition prepared as described above is about 100 to about 500 mcg of tilmanocept, depending on the patient's age and weight, totaling 1 cc. The isoniazid-tilmanocept composition is administered to the patient by intravenous injection. When administered in this fashion, the isoniazid-tilmanocept composition would be expected to localize in the granulomas containing the intracellular tuberculosis bacilli and deliver the isoniazid to the intracellular space within the macrophage where the TB is located. This will allow for a concentrated dose of Isoniazid to be delivered directly to the tuberculosis bacilli, bypassing the usual barriers of drug delivery frequently encountered in TB treatment.

In variations of the above TB treatment composition and method, indomethacin and/or Ga (optionally as ⁶⁸Ga) are also attached to the tilmanocept in the manner described previously in order to provide Ga-isoniazid-tilmanocept, indomethacin-isoniazid-tilmanocept, and/or indomethacin-Ga-isoniazid-tilmanocept, which is then formulated into suitable compositions and administered in the various manners described previously. As a still further variation, Ga (optionally as ⁶⁸Ga) and/or Ga-isoniazid-tilmanocept is attached to the tilmanocept in place of isoniazid in the manner described previously in order to provide Ga-tilmanocept, indomethacin-tilmanocept and/or Ga-indomethacin-tilmanocept which is then formulated in to suitable compositions and administered in the various manners described previously.

### Diagnosis of Increased Arterial Inflammation

Macrophages, in high-risk coronary atherosclerotic plaque samples from patients who experienced sudden cardiac death, express CD206—along with CD163. These high-risk plaques have been characterized, morphologically, as thin-cap fibroatheromas (TCFA) and infiltrated by activated macrophages throughout the necrotic plaque core and thin, fibrous plaque cap. Thus, increased arterial inflammation, as evidenced by the presence of macrophages, is an indicator of increased risk for developing high risk morphology coronary plaque burden.

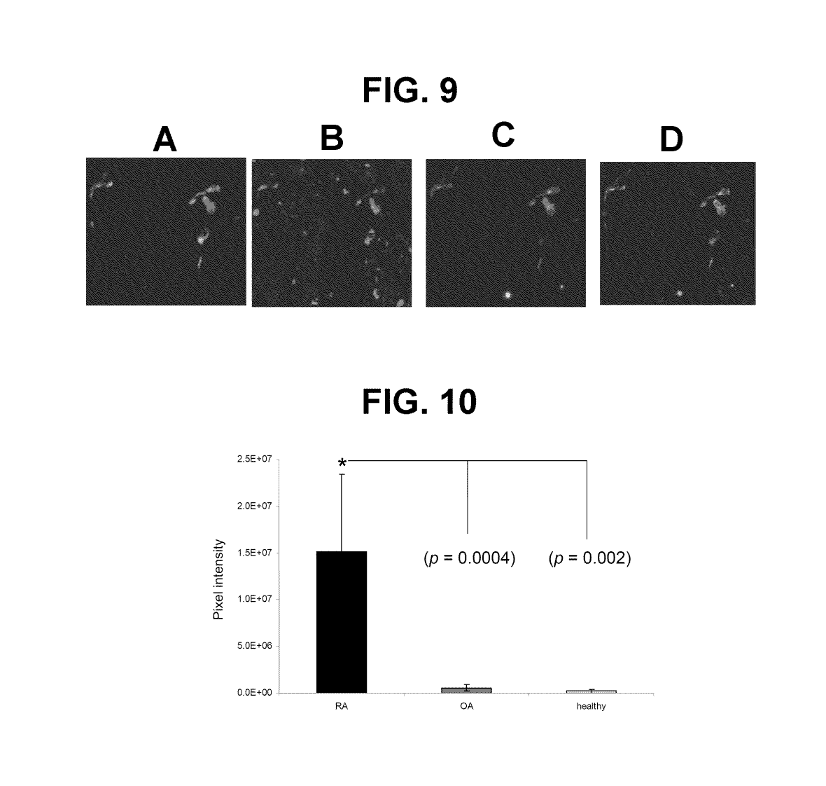

FIG. 9 depicts images of immunofluorescent staining of left ventricle and aorta from rhesus macaque. The images illustrate the Co-localization of CD163/Alex-Fluor 488, CD206/Alexa-Fluor 568, and Cy3 tilmanocept. Alexa-Flour 568 is fluorescent dye readily attachable to tilmanocept in the manner previously described.

Based on their unique properties to tag activated macrophages, the compositions of the present invention provide a method for imaging arterial inflammation and identifying individuals with arterial macrophage-specific inflammation and heightened immune-mediated cardiovascular disease (CVD) risk.

One embodiment of the present invention provides a method of quantifying measurable aortic uptake of the of systemically injected CD206 targeting compositions of the present invention using single photon emission computed tomography (SPECT/CT). In another embodiment, the present invention provides a method of measuring the density of infiltrating activated macrophages in arterial atherosclerotic plaque.

In another embodiment, the invention provides a method of functional arterial imaging to characterize the propensity of individual coronary plaques to rupture. In another embodiment, the invention provides a method for identifying patients at risk for CVD before they experience clinically significant events. In one particular embodiment, the method is applied to specific high-risk patients, such as HIV-infected patients. In another particular embodiment, the method is applied to patients with vulnerable plaque at risk for rupture in the general population. F In another particular embodiment, the invention provides functional arterial imaging for monitoring the efficacy of anti-inflammatory strategies that modulate accelerated atherogenesis.

### Arterial Inflammation Imaging Example

The following example illustrates the evaluation of aortic and coronary artery 99 mTc-tilmanocept uptake using SPECT and SPECT/CT Imaging).

An 18 year-old male patient, with documented HIV infection with a history of subclinical aortic plaque and high-risk morphology coronary plaque on CCTA, receives an intravenous injection via catheter of ˜10 mCi of 99 mTc-tilmanocept. The catheter is flushed post-injection with approximately 10 mL of saline solution.

The subject is positioned on the scanner table of a Siemens SPECT/CT scanner (Siemens Medical Solutions, Hoffman Estates, Ill.). After a lag time of approximately 60 minutes, SPECT acquisitions will be performed using 2×60 views, step and shoot mode, 1 min per view of the thorax and neck based on the scout CT performed prior to SPECT. Gated acquisitions are performed when imaging the heart. Thoracic images include all of the lungs. Data acquisition of SPECT and CT take approximately 70 minutes. All projections are acquired in two energy windows, namely [90-120 keV] and [126-154 keV], corrected for Compton scatter using a scatter window and for attenuation using the CTAC and reconstructed using iterative ordered subsets expectation maximization algorithm (OSEM). The resulting reconstructed volume are used to quantify target to background ration using regions of interest (ROI) drawn on areas of 99 mTc-tilmanocept uptake of interest normalized to a reference region of interest

### Diagnosis of Rheumatoid Arthritis

Rheumatoid arthritis (“RA”) is an autoimmune disease that is also difficult to diagnose and treat. The carrier molecules described herein (e.g., tilmanocept) are designed to bind to CD206 of reticuloendothelial cells that are invasive to focal RA tissue, such as is present in association with RA of the joints and/or viscerally-involved. Thus these carrier molecules can be used for diagnostic imaging as well as treatment of RA. As to tilmanocept, for example, the mannoses act as ligand moieties for the CD206, and the DTPA serves as a chelating moiety for radiolabeling with, for example, Tc 99m. When Tc 99m-tilmanocept is injected in close proximity to the suspected diseased locus (i.e., a joint where RA is present or suspected), scintigraphic imaging in conjunction with a stationary gamma camera and/or intraoperatively in conjunction with a handheld gamma detection probe may be used to localize involved tissue for purposes of diagnosing RA. Tilmanocept has a mean diameter of about 7 nm, and this small diameter permits enhanced diffusion into tissue channels and blood capillaries, resulting in a rapid injection site clearance and CD206 binding in the inflammasome. By way of example, fluorescent and/or radioactive tilmanocept and other carrier molecules described previously can be used for the diagnosis and non-invasive imaging of joints with early forms of RA.

In order to demonstrate that tilmanocept binds to CD206 receptors found on macrophages in synovial fluid of subjects having RA, synovial fluid and tissue were acquired from patients diagnosed with frank RA. Tissues were probed with Manocept-Cy3, DAPI nuclear fluor, and anti CD206-cyanine green. The tissues and fluids were imaged by micro-fluorescence and compared to normal frozen archival tissue and synovial tissue procured from patients with osteoarthritis (OA). MP localization and degree of fluorescence were compared by digital image analysis. In particular, the micro-fluoroescence images were analyzed using scanning quantitative fluorescence microscopy, with integration algorithms was used to quantitate and contrast pixel counts of Cy3 fluorescent dye in images of tissue and synovial fluid.

The results indicated that the synovial tissue and fluid from subjects with RA contain large macrophage populations that express high levels of CD206. Additionally, these MPs strongly localize Cy3-tilmanocept on CD206. In addition, the degree of macrophage invasion and CD206 residence in normal and OA tissue is significantly lower than in RA tissues, as seen in FIG. 10. Thus, the carrier molecules of the present invention, when provided with a detectable moiety such as a fluorophore, are able to not only diagnose RA from synovial fluid (either in vivo or ex vivo), but also can distinguish RA from OA.

Imaging of macrophages in cartilage antibody-induced arthritis in mice using Cy3-tilmanocept

Cy3-tilmanocept was used to image macrophages in a mouse model of early immune-mediated arthritis and cartilage antibody-induced arthritis in Dba1 mice using fluorescent luminescence. Arthritis was induced in mice by injection of a five monoclonal antibody anti-cartilage cocktail followed in three days by an injection of E. coli lipopolysaccharide. The mice developed swollen and reddened joints in the feet, carpi, tarsi, elbows, and knees of variable degrees in 7-11 days, evidencing arthritis.

Mice were imaged in vivo on days 7 or 8 and mice were euthanized on days 9 or 11. After euthanasia, the limbs were dissected, skin was removed, and the samples were reimaged (epifluorescent imaging), radiographed (Faxitron MX20) and then decalcified, embedded, and stained with H&E.

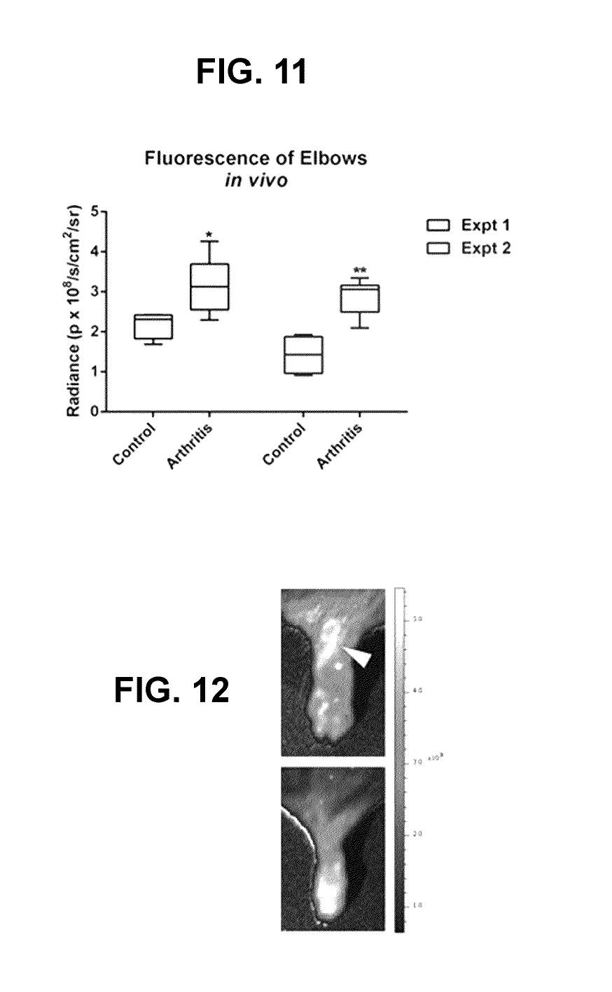

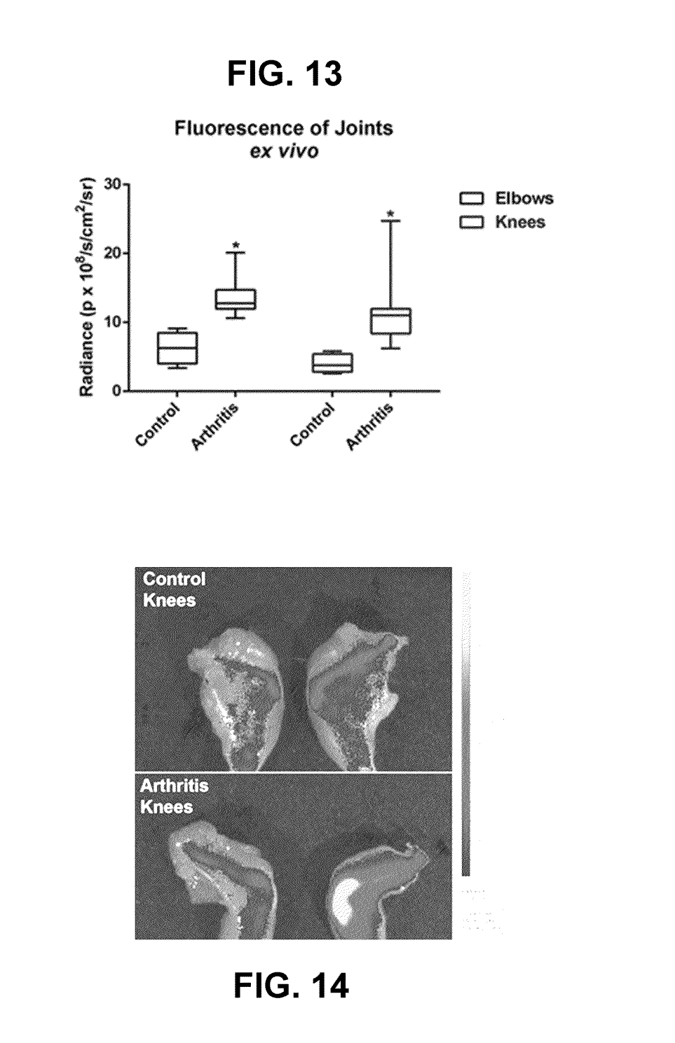

For epifluorescent imaging, mice were injected intravenously with Cy3-tilmanocept, and epifluorescent imaging was conducted in vivo and ex vivo at 1-2 hours using an IVIS Lumina II machine (Caliper Life Sciences, Hopkinton, Mass.). Living Image software was used to visualize the visible and fluorescent images and to quantitate the number of photons using regions of interest (“ROI”) and subtraction of background fluorescence. After euthanasia the limbs were dissected, skin was removed (except for the digits), and re-imaged. Specific fluorescence was detected in arthritic knees and elbows, as seen in FIG. 11. FIG. 12 depicts in vivo fluorescence of the elbows and feet of a mouse with immunemediated arthritis (top) and control mouse (bottom). The mouse with arthritis had increased fluorescence due to Cy3-Tilmanocept in the elbow compared to the control mouse. There was background fluorescence from the skin, which was prominent on the feet. FIG. 13 shows ex vivo fluorescence data, and FIG. 14 depicts ex vivo fluorescence of the knees of control mice and mice with immune-mediated arthritis. Although both knees in the treated mouse (lower image) had arthritis, the knee on the right was affected more severely and had greater fluorescence due to Cy3-Tilmanocept labeling.

In particular embodiments for RA diagnostic imaging, the carrier molecule is tilmanocept, and the detectable moiety is ^{99m}Tc or ⁶⁸Ga attached to the DTPA chelator prior to use, or a fluorescent dye attached to the amino-terminated leash (e.g., Cy3). In the case of Cy3-tilmanocept, optical imaging is employed at to determine the presence and/or extend of RA. The above-described mouse studies have confirmed that labeled tilmanocept (e.g., Cy-3-tilmanocept) is useful in diagnosing RA.

In particular embodiments for RA treatment, the carrier molecule is tilmanocept, and the therapeutic is a therapeutic isotope. In one particular embodiment, the therapeutic isotope is ^{117m}Sn. In other particular embodiments for RA treatment, the carrier molecule is tilmanocept, and the therapeutic is a toxin. In one particular embodiment, the toxin is botulinum or cholera toxin. In another particular embodiment for RA treatment, the carrier molecule is tilmanocept, and the therapeutic is a methotrexate.

### RA Imaging Example

Tilmanocept lyophilized powder, marketed by Navidea Biopharmaceuticals Inc. under the name LYMPHOSEEK® Injection kit, is obtained. The tilmanocept powder has a mean diameter of about 7 nm, and is contained in a 0.5 mL vial as a mixture of 0.250 mg tilmanocept, 20 mg trehalose dihydrate, 0.5 mg glycine, 0.5 mg sodium ascorbate and 0.075 mg stannous chloride dihydrate. The tilmanocept powder is then radiolabeled with Tc 99m using sodium 99 mTc-pertechnetate eluted from a Technetium-99m generator. Using a sterile syringe, approximately 92.5 MBq (2.5 mCi) of sodium 99 mTc-pertechnetate in about 0.35 mL is aseptically added to the vial. The vial is gently shaken, and the radiolabeling reaction allowed to proceed at room temperature for at least 10-15 minutes. Normal saline is then added to the vial to bring the contents to 5 cc, and a buffer may optionally be added as described previously.

A single patient dose is 100 mcg of tilmanocept and 1 mCi of technetium 99m, totaling 2 cc. The radiolabeled tilmanocept is administered by intravenous injection within six hours of radiolabeling. Within 30 to 180 minutes of injection, the patient is imaged using Single Photon Emission Computed Tomography (SPECT). The findings of localized radioactivity in the joints is presumptive evidence of an inflammatory process in the intrarticular area, which is a hallmark of rheumatoid arthritis (RA), thereby ruling out RA if it is absent and aiding in the diagnosis of early or ongoing RA if it is present.

While several compositions and methods for the diagnosis and/or treatment of macrophage-related disorders have been discussed in detail above, it should be understood that the compositions, features, configurations, and methods of using the compositions discussed are not limited to the contexts provided above.

What is claimed is:

1. A method of diagnosing a CD206 expressing cell-related disorder comprising the steps of:
(a) administering a pharmaceutical composition to a subject, said composition including a carrier molecule having a detectable moiety attached thereto, said carrier molecule comprising:
i. a dextran backbone; and
ii. at least one receptor substrate conjugated, directly or indirectly, to said dextran backbone, said at least one receptor substrate chosen so as to specifically bind to CD206;
wherein said carrier molecule is water soluble; and
(b) after said administering step, detecting the presence of said detectable moiety at a location in the subject other than a sentinel lymph node.

2. The method of claim 1, wherein said receptor substrate is chosen from the group consisting of a residue of mannose, fucose, n-acetylglucosamine, D-galactose, n-acetylgalactoseamine, sialic acid and neuraminic acid.

3. The method of claim 2, wherein said receptor substrate comprises two or more residues selected from the group consisting of mannose, fucose, n-acetylglucosamine, D-galactose, n-acetylgalactoseamine, sialic acid and neuraminic acid, conjugated to a single glucose moiety of the dextran backbone.

4. The method of claim, 2 wherein said carrier molecule has at least one leash, and at least one of said receptor substrate and said detectable moiety is attached to the dextran backbone via said leash.

5. The method of claim 4 wherein said leash is —O(CH₂)₃S(CH₂)₂NH₂.

6. The method of claim 1, wherein said detecting step comprises quantitating the level of the detectable moiety in tissue at a predetermined location associated with the CD206 expressing cell-related disorder being diagnosed.

7. The method of claim 1, wherein the CD206 expressing cell-related disorder is an inflammatory disorder

8. The method of claim 7, wherein the CD206 expressing cell-related disorder is an angiogenic disorder.

9. The method of claim 7, wherein the CD206 expressing cell-related disorder is cancer, tuberculosis, HIV, Kaposi's sarcoma, Alzheimer's disease, rheumatoid arthritis, or multiple sclerosis.

10. The method of claim 1, wherein said at least one receptor substrate is a polysaccharide.

11. The method of claim 11, wherein the polysaccharide is mannan.

12. The method of claim 1, wherein the detectable moiety is a fluorophore.

13. The method of claim 12, wherein the detectable moiety is Cy-3.

14. The method of claim 1, wherein the detectable moiety is a radioisotope.

15. The method of claim 14, wherein the detectable moiety is ⁶⁸Ga.

16. The method of claim 14, wherein the detectable moiety is ^{99m}Tc.

17. A method of treating a CD206 expressing cell-related disorder comprising the steps of:
(a) administering a therapeutically effective amount of a pharmaceutical composition to a subject, said composition including a carrier molecule having a therapeutic agent attached thereto, said carrier molecule comprising:
i. a dextran backbone; and
ii. at least one receptor substrate conjugated, directly or indirectly, to said dextran backbone, said at least one receptor substrate chosen so as to specifically bind to CD206;
wherein said carrier molecule is water soluble.

18. The method of claim 17, wherein said receptor substrate is chosen from the group consisting of a residue of mannose, fucose, n-acetylglucosamine, D-galactose, n-acetylgalactoseamine, sialic acid and neuraminic acid.

19. The method of claim 18, wherein said receptor substrate comprises two or more residues of mannose, fucose, n-acetylglucosamine, D-galactose, n-acetylgalactoseamine, sialic acid and neuraminic acid, conjugated to a single glucose moiety of the dextran backbone.

20. The method of claim 17, wherein said carrier molecule has at least one leash, and at least one of said receptor substrate and said therapeutic agent is attached to the dextran backbone via said leash.

21. The method of claim 20 wherein said leash is —O(CH₂)₃S(CH₂)₂NH₂.

22. The method of claim 17, wherein the CD206 expressing cell-related disorder is an inflammatory disorder.

23. The method of claim 22, wherein the CD206 expressing cell-related disorder is an angiogenic disorder.

24. The method of claim 22, wherein the CD206 expressing cell-related disorder is cancer, tuberculosis, HIV, Kaposi's sarcoma, Alzheimer's disease, rheumatoid arthritis, vulnerable plaque thin-fibro-atheroma, or multiple sclerosis.

25. The method of claim 17, wherein said at least one receptor substrate is a polysaccharide.

26. The method of claim 25, wherein the polysaccharide is mannan.

27. The method of claim 17, wherein the CD206 expressing cell-related disorder is rheumatoid arthritis.

28. The method of claim 17 wherein the CD206 expressing cell-related disorder is Kaposi's sarcoma.

29. The method of claim 1, wherein the CD206 expressing cell-related disorder is rheumatoid arthritis.

30. The method of claim 1 wherein the CD206 expressing cell-related disorder is Kaposi's sarcoma.

31. A method of diagnosing and/or treating tuberculosis comprising the steps of:
(a) administering a pharmaceutical composition to a subject, said composition including a carrier molecule having a detectable moiety and/or therapeutic agent attached thereto, said carrier molecule comprising:
i. a dextran backbone;
ii. at least one receptor substrate conjugated, directly or indirectly, to said dextran backbone, said at least one receptor substrate chosen so as to specifically bind to CD206; and
iii. at least one radioactive isotope or cytotoxic agent conjugated, directly or indirectly, to said dextran backbone; and
(b) optionally, after said administering step, detecting the presence of said radioactive isotope in the subject's lung tissue.

32. The method of claim 31, wherein the receptor substrate is chosen from the group consisting of a residue of mannose, fucose, n-acetylglucosamine, D-galactose, n-acetylgalactoseamine, sialic acid, and neuraminic acid.

33. The method of claim 31, wherein the receptor substrate is mannan.

34. The method of claim 31, wherein ⁶⁸Ga is conjugated to said dextran backbone.

35. An ex vivo method for quantitating the number of cells expressing CD206 in a bodily fluid obtained from a mammalian subject, comprising the steps of:
(a) contacting the bodily fluid obtained from the mammalian subject with a carrier molecule having at least one detectable moiety attached thereto such that the carrier molecule binds to cells expressing CD206 which are present in the bodily fluid, the carrier molecules comprising:
i. a dextran backbone, and
ii. at least one receptor substrate conjugated, directly or indirectly, to said dextran backbone, said at least one receptor substrate chosen so as to specifically bind to CD206,
wherein said carrier molecule is water soluble;
(b) separating insoluble cells from unbound carrier molecules to provide a cell fraction; and
(d) measuring the level of detectable moiety in the cell fraction in order to quantitate the number of cells expressing CD206.

36. The method of claim 35, wherein the step of contacting the bodily fluid with the carrier molecule comprises combining the bodily fluid and carrier molecule in a container, and thereafter incubating the resulting mixture for a predetermined period of time.

37. The method of claim 36, wherein said resulting mixture is incubated at a temperature of between about 0° C. and about 25° C.

38. The method of claim 36, wherein said resulting mixture is incubated at a temperature of about 4° C.

39. The method of claim 36, wherein the step of separating insoluble cells from unbound carrier molecules comprises centrifuging the mixture of bodily fluid and carrier molecules.

40. The method of claim 35, wherein the detectable moiety comprises at least one a fluorophore, and said step of measuring the level of detectable moiety in the cell fraction comprises spectroscopically measuring the level of fluorescence of the cell fraction.

41. The method of claim 35, wherein said carrier molecule comprises tilmanocept.

42. The method of claim 35, wherein said fluorophore is Cy3.

43. The method of claim 35, wherein said bodily fluid comprises synovial fluid.

44. A diagnostic kit for quantitating the number of cells expressing CD206 in a bodily fluid obtained from a mammalian subject, comprising:
(a) a first sealed container containing a carrier molecule with at least one spectroscopically detectable moiety attached thereto, the carrier molecule comprising:
i. a dextran backbone, and
ii. at least one receptor substrate conjugated, directly or indirectly, to said dextran backbone, said at least one receptor substrate chosen so as to specifically bind to CD206,
wherein said carrier molecule is water soluble;
(b) a second sealed container containing a diluent;
(c) at least one centrifuge vial; and
(d) at least one cuvette.

45. The diagnostic kit of claim 44, wherein said diluent is sterile saline or a buffered diluent solution.
